(19) United States
(12) Patent Application Publication
(10) Pub. No.: US 2012/0282168 A1
(43) Pub. Date: Nov. 8, 2012

(54) METHODS OF SENSITIZING CANCER TO THERAPY-INDUCED CYTOTOXICITY

(75) Inventors: Benjamin BONAVIDA, Los Angeles, CA (US); Michael PALLADINO, Encinitas, CA (US)

(73) Assignees: Nereus Pharmaceuticals, Inc., San Diego, CA (US); The Regents of the University of California, Oakland, CA (US)

(21) Appl. No.: 13/457,293

(22) Filed: Apr. 26, 2012

Related U.S. Application Data
(63) Continuation of application No. 12/282,343, filed on Feb. 19, 2009, now abandoned.
(60) Provisional application No. 60/733,965, filed on Nov. 4, 2005.
(60) Provisional application No. 60/840,811, filed on Aug. 28, 2006.

(51) Int. Cl. A61K 31/407; A61P 35/00; A61K 31/69; A61K 51/00; A61K 39/395

(52) U.S. Cl. 424/1.11; 514/421; 424/133.1; 514/64

(57) ABSTRACT

The present application demonstrates that Salinosporamide A can be used to sensitize cancer cells to cancer therapy. Furthermore, the present application demonstrates that Salinosporamide A acts as a therapeutic agent to kill or inhibit cancer cells after sensitization of the cells by an antibody or other chemosensitizing reagents. The cancer cells can be either therapy-sensitive or therapy resistant. The present application further demonstrates that Salinosporamide A induces the expression of Raf kinase inhibitor protein (RKIP) and PTEN, tumor suppressor proteins, and inhibits the expression of YY1, a transcriptional regulator protein overexpressed in cancer cells and also inhibits the growth factor pleiotrophin (PTN).

### CROSS-REFERENCES TO RELATED APPLICATIONS

This application claims priority to U.S. Ser. No. 60/733,965, filed on Nov. 4, 2005, and U.S. Ser. No. 60/840,811, filed Aug. 28, 2006, the teachings of which are incorporated herein by reference.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

Proteasome inhibitors have been shown to induce cell killing alone and/or in combination with drugs in drug-resistant tumor cells. In 2003, the FDA approved the first proteasome inhibitor VELCADE, “bortezomib” for treating patients with multiple myeloma who relapsed after two therapies and are progressing on current treatments. Thus, proteasome inhibitors prove to be clinically effective. However, like many other drugs, resistance to bortezomib starts to emerge as well as bortezomib-induced tissue toxicity has been noted. The development of new proteasome inhibitors that can override bortezomib resistance and exhibiting less toxicity is highly desirable. The chemical compound Salinosporamide A (NPI-0052, Nereus Pharmaceuticals, San Diego) was discovered during the fermentation of Salinospora species, a new marine gram positive actinomycete. It is related to two less potent 20S proteasome inhibitors, structurally related to lactacystin, omuralide, and PS-519.

Several in vitro findings indicated that Salinosporamide A exhibited cytotoxicity against a variety of tumor cell lines (Feling, et al., Angew. Chem. Int. Ed., 2003, 42(3): 355-357) and can exert apoptosis and inhibition of NF-κB (Macherla, et al., Journal of Medicinal Chemistry, 2005, 48:3684). It has also been shown that Salinosporamide A is effective in bortezomib-resistant cell lines. In vivo, Salinosporamide A exerted anti-tumor effects whether administered orally or intravenously (Chuahan et al., Cancer Cell, Nov. 8, 407-419 (2005)). Salinosporamide A has been synthesized chemically. Studies on cytotoxicity with the NCI screening panels of 60 human tumor cell lines showed that Salinosporamide A affected many cancer cells, and had a mean growth inhibition of less than 10 nM. Other tumor cell lines examined showed significant cytotoxic activity. Noteworthy, Salinosporamide A was also cytotoxic to both drug sensitive HL60 and drug resistant HL60MX2 with equal doses.

Salinosporamide A also had the effect of inducing a range of direct apoptosis on different tumor cell lines. The effect of Salinosporamide A on the induction of apoptosis suggests that Salinosporamide A may be used as an agent to identity anti-apoptotic pathways that may serve as targets for cancer therapy by examining changes in the expression of nucleic acids and proteins upon the treatment of cancer cells with this compound.

In the present application, we have examined if Salinosporamide A can sensitize therapy sensitive and therapy-resistant B Non-Hodgkin's Lymphoma to therapy-induced apoptosis. We also investigated whether Salinosporamide A could act as a therapeutic agent and induce apoptosis after sensitization by another compound such as rituximab. Furthermore, we have also examined the effect of Salinosporamide A on the induction of Raf kinase inhibitor protein (RKIP), a metastasis tumor suppressor protein that potentiates anti-apoptotic pathways in cancer cells, and on the inhibition of expression of YY1, a transcriptional regulator protein overexpressed in cancer cells that regulates tumor cell resistance to both chemotherapy and immunotherapy

### BRIEF SUMMARY OF THE INVENTION

The present application demonstrates that Salinosporamide A, in combination with subtoxic therapeutically effective amounts of cancer therapeutic agents, sensitizes both resistant and sensitive cancer cells to therapy-induced cytotoxicity. The cancer cells can be either therapy-sensitive or therapy resistant. Furthermore, the present application demonstrates that Salinosporamide A acts as a therapeutic agent to induce apoptosis in cancer cells after sensitization of the cells by an antibody or by various chemo- and immuno sensitizing agents. The cancer cells can be either therapy-sensitive or therapy resistant. Additionally, the present application demonstrates that Salinosporamide A induces the expression of RKIP, thereby inhibiting survival anti-apoptotic signaling pathways and resulting in reducing the threshold of anti-apoptotic gene expression and when used alone, or in combination with other agents, results in apoptosis. Furthermore, induction of RKIP also exerts anti-angiogenic activity as well as prevents metastasis. Further Salinosporamide A treatment inhibits the transcription repressor YY1, resulting in the upregulation of death receptors and sensitization of tumor cells to cytotoxic immunotherapy. It also regulates death receptor expression in rituximab-resistant clones. Salinosporamide A-induces the expression of the AKT inhibitor PTEN resulting in downstream inhibition of the AKT anti-apoptotic and survival pathway and resulting in inhibition of anti-apoptotic gene products Salinosporamide A also inhibits the overexpression of pleiotrophin (PTN) a growth factor and resistance factor in tumor cells and circulating levels of PTN have been shown to have a prognostic importance.

In a first embodiment, the invention provides a method of treating, preventing or inhibiting a cancer by administering to a subject a therapeutically effective amount of a cancer therapy reagent and a sensitizingly effective amount of a compound of Formula I:

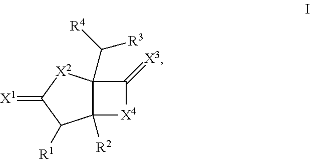

in which each of R¹, R² and R³ can independently be: H, alkyl, substituted alkyl, alkenyl, substituted alkenyl, alkynyl, substituted alkynyl, aryl, substituted aryl, heteroaryl, substituted heteroaryl, heterocyclic, substituted heterocyclic, cycloalkyl, substituted cycloalkyl, alkoxy, substituted alkoxy, thioalkyl, substituted thioalkyl, hydroxy, halogen, amino, amido, carboxyl, —C(O)H, acyl, oxyacyl, carbamate, sulfonyl, sulfonamide, or sulfuryl; R⁴ is a 5-8 membered cycloalkyl optionally substituted with 1-8 R⁵ groups or a 5-8 membered cycloalkenyl optionally substituted with 1-8 R⁵ groups; each R⁵ can independently be: alkyl, substituted alkyl, alkenyl, substituted alkenyl, alkynyl, substituted alkynyl, aryl, substituted aryl, cycloalkyl, or substituted cycloalkyl; each of X¹, X², X³ and X⁴ can be independently: O, NR⁶ and S; and R⁶ is H or C₁-C₆ alkyl.

In some aspects of the first embodiment, each of R¹ and R² can independently be: H, alkyl, substituted alkyl, alkenyl, substituted alkenyl, alkynyl, substituted alkynyl, cycloalkyl, and substituted cycloalkyl; R³ can be alkoxy, substituted alkoxy, thioalkyl, substituted thioalkyl, hydroxy, halogen, amino, amido, oxyacyl, carbamate, sulfonyl, sulfonamide, or sulfuryl; R⁴ is a 5-8 membered cycloalkenyl optionally substituted with 1-8 R⁵ groups; each of X¹, X³ and X⁴ is O; and X² is NH.

In yet another aspect of the first embodiment, each of R¹ and R² can independently be: alkyl, substituted alkyl, alkenyl, substituted alkenyl, alkynyl, and substituted alkynyl; R³ can be alkoxy, substituted alkoxy, thioalkyl, substituted thioalkyl, hydroxy, and amino; R⁴ is cyclohexenyl optionally substituted with 1-8 R⁵ groups; each of X¹, X³ and X⁴ is O; and X² is NH.

In further aspects of the first embodiment, R¹ is an alkyl or substituted alkyl; R² is alkyl; R³ is hydroxy; R⁴ is cyclohexenyl; and each of X¹, X³ and X⁴ is O; and X² is NH; the substituted alkyl of R¹ can be a halogenated alkyl, which can be fluorinated, chlorinated, brominated in different aspects. In some aspects, the halogenated alkyl compound has the following structure:

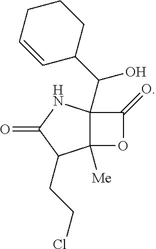

In yet another aspect of the first embodiment, the halogenated alkyl compound has the following structure:

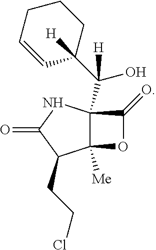

In further aspects of the first embodiment, the sensitizingly effective amount of the compound of Formula I is sufficient to induce expression of RKIP or PTEN, thereby inducing or facilitating apoptosis. The expression of RKIP or PTEN can be at least 1, 2, 10, or 100 fold higher than in the absence of the compound of Formula I.

In yet further aspects of the first embodiment, the sensitizingly effective amount of the compound of Formula I is sufficient to inhibit the expression of YY1, and PTN, thereby inducing apoptosis. The expression of YY1, PTEN, and PTN can be at least 1, 2, 10, or 100 fold lower than in the absence of the compound of Formula I.

In another aspect of the first embodiment, the cancer therapy reagent can be a chemotherapeutic reagent, an immunotherapeutic reagent, a radiotherapeutic reagent, a hormonal therapeutic reagent, or a pharmacologic inhibitor.

In other aspects of the first embodiment, the cancer can be non-Hodgkin's lymphoma, B-acute lymphoblastic lymphoma, prostate cancer, ovarian cancer, renal cancer, lung cancer, breast cancer, colon cancer, leukemia, multiple myeloma and hepatocarcinoma.

In another aspect of the first embodiment, the cancer therapy reagent induces or facilitates apoptosis and can be a chemotherapeutic reagent, an immunotherapeutic reagent, a radiotherapeutic reagent, a hormonal therapeutic reagent, or a pharmacologic inhibitor. In this aspect, the cancer therapy reagent can be rituximab immunotherapy.

In various aspects of the first embodiment, the cancer is therapy-resistant, including resistance to immunotherapy, chemotherapy, radiotherapy, or hormonal therapy. However, in other aspects, the cancer can be therapy-sensitive.

In further aspects of the first embodiment, the therapeutically effective amount of a cancer therapy reagent and the sensitizingly effective amount of a compound of Formula I are administered concurrently or sequentially, in which the cancer therapy reagent is bortezomib administration. In related aspects, the cancer therapy reagent can be a chemotherapeutic reagent, an immunotherapeutic reagent, a radiotherapeutic reagent, a hormonal therapeutic reagent, or a pharmacologic inhibitor.

In an alternative aspect of the first embodiment, the therapeutically effective amount of a cancer therapy reagent and the sensitizingly effective amount of a compound of Formula I are administered sequentially.

In an aspect of the first embodiment, the subject can be a human.

A second embodiment of this invention provides a method of treating, preventing or inhibiting lymphoma by administering to a subject a therapeutically effective amount of a cancer therapy reagent and a sensitizingly effective amount of a compound of Formula I:

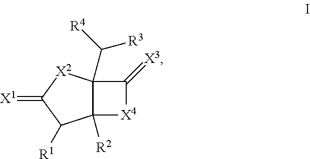

in which each of R¹, R² and R³ can independently be: H, alkyl, substituted alkyl, alkenyl, substituted alkenyl, alkynyl, substituted alkynyl, aryl, substituted aryl, heteroaryl, substituted heteroaryl, heterocyclic, substituted heterocyclic, cycloalkyl, substituted cycloalkyl, alkoxy, substituted alkoxy, thioalkyl, substituted thioalkyl, hydroxy, halogen, amino, amido, carboxyl, —C(O)H, acyl, oxyacyl, carbamate, sulfonyl, sulfonamide, or sulfuryl; R⁴ is a 5-8 membered cycloalkyl optionally substituted with 1-8 R⁵ groups or a 5-8 membered cycloalkenyl optionally substituted with 1-8 R⁵ groups; each R⁵ can independently be: alkyl, substituted alkyl, alkenyl, substituted alkenyl, alkynyl, substituted alkynyl, aryl, substituted aryl, cycloalkyl, and substituted cycloalkyl; each of X¹, X², X³ and X⁴ is independently selected from the group consisting of O, NR⁶ and S; and R⁶ is H or C₁-C₆ alkyl.

In an aspect of the second embodiment, the sensitizingly effective amount of the compound of Formula I is sufficient to induce expression of RKIP or PTEN, thereby inducing or potentiating apoptosis.

In another aspect of the second embodiment, the sensitizingly effective amount of the compound of Formula I is sufficient to inhibit the expression of YY1 or PTN thereby inducing or potentating apoptosis.

In further aspects of the second embodiment, the lymphoma is therapy resistant, which can include a lymphoma which is rituximab therapy resistant.

In a third embodiment, this invention provides a method of treating, preventing or inhibiting lymphoma by administering to a subject a therapeutically effective amount of rituximab and a sensitizingly effective amount of a compound of Formula I:

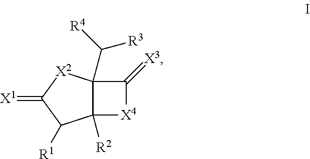

in which each of R¹, R² and R³ can independently be: H, alkyl, substituted alkyl, alkenyl, substituted alkenyl, alkynyl, substituted alkynyl, aryl, substituted aryl, heteroaryl, substituted heteroaryl, heterocyclic, substituted heterocyclic, cycloalkyl, substituted cycloalkyl, alkoxy, substituted alkoxy, thioalkyl, substituted thioalkyl, hydroxy, halogen, amino, amido, carboxyl, —C(O)H, acyl, oxyacyl, carbamate, sulfonyl, sulfonamide, or sulfuryl; R⁴ is a 5-8 membered cycloalkyl optionally substituted with 1-8 R⁵ groups or a 5-8 membered cycloalkenyl optionally substituted with 1-8 R⁵ groups; each R⁵ can independently be: alkyl, substituted alkyl, alkenyl, substituted alkenyl, alkynyl, substituted alkynyl, aryl, substituted aryl, cycloalkyl, and substituted cycloalkyl; each of X¹, X², X³ and X⁴ can independently be O, NR⁶ and S; and R⁶ is H or C₁-C₆ alkyl.

In an aspect of the third embodiment, the sensitizingly effective amount of the compound of Formula I is sufficient to induce expression of RKIP or PTEN, thereby inducing apoptosis.

In another aspect of the third embodiment, the sensitizingly effective amount of the compound of Formula I is sufficient to inhibit expression of YY1 or PTN, thereby inducing apoptosis.

In a fourth embodiment, the invention provides a composition containing a therapeutically effective amount of rituximab and a sensitizingly effective amount of a compound of Formula I in a physiologically acceptable excipient.

In a fifth embodiment, the invention provides a kit comprising a therapeutically effective amount of rituximab and a sensitizingly effective amount of a compound of Formula I.

In a sixth embodiment, this invention provides a method of treating, preventing or inhibiting a cancer with proteasome inhibitor therapy by administering to a subject a sensitizingly effective amount of an antibody or chemosensitizing reagent and a therapeutically effective amount of a compound of Formula I:

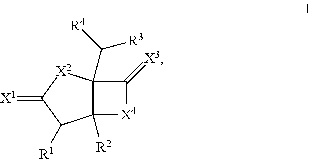

in which each of R¹, R² and R³ can independently be: H, alkyl, substituted alkyl, alkenyl, substituted alkenyl, alkynyl, substituted alkynyl, aryl, substituted aryl, heteroaryl, substituted heteroaryl, heterocyclic, substituted heterocyclic, cycloalkyl, substituted cycloalkyl, alkoxy, substituted alkoxy, thioalkyl, substituted thioalkyl, hydroxy, halogen, amino, amido, carboxyl, —C(O)H, acyl, oxyacyl, carbamate, sulfonyl, sulfonamide, or sulfuryl; R⁴ is a 5-8 membered cycloalkyl optionally substituted with 1-8 R⁵ groups or a 5-8 membered cycloalkenyl optionally substituted with 1-8 R⁵ groups; each R⁵ can independently be: alkyl, substituted alkyl, alkenyl, substituted alkenyl, alkynyl, substituted alkynyl, aryl, substituted aryl, cycloalkyl, and substituted cycloalkyl; each of X¹, X², X³ and X⁴ can independently be O, NR⁶ and S; and R⁶ is H or C₁-C₆

In an aspect of the sixth embodiment, the sensitizingly effective amount of the compound of Formula I is sufficient to induce expression of RKIP or PTEN, thereby inducing apoptosis or sensitizing cells to apoptosis by various sub-toxic concentrations on cytotoxic agents. In other aspects of this embodiment, the antibody is rituximab and the cancer is lymphoma.

In another aspect of the sixth embodiment, the sensitizingly effective amount of the compound of Formula I is sufficient to inhibit expression of YY1 or PTN, thereby inducing apoptosis. In other aspects of this embodiment, the antibody is rituximab and the cancer is lymphoma.

In a seventh embodiment, this invention provides a composition comprising a sensitizingly effective amount of rituximab and a therapeutically effective amount of a compound of Formula I in a physiologically acceptable excipient.

In an eighth embodiment, this invention provides a kit comprising a sensitizingly effective amount of rituximab and a therapeutically effective amount of a compound of Formula I.

In a ninth embodiment, this invention provides a method of treating, preventing or inhibiting a cancer, the method comprising the step of administering to a subject a therapeutically effective amount of a compound of Formula I:

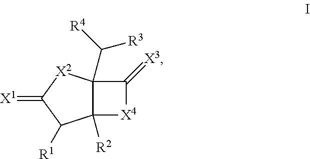

in which each of R¹, R² and R³ can independently be: H, alkyl, substituted alkyl, alkenyl, substituted alkenyl, alkynyl, substituted alkynyl, aryl, substituted aryl, heteroaryl, substituted heteroaryl, heterocyclic, substituted heterocyclic, cycloalkyl, substituted cycloalkyl, alkoxy, substituted alkoxy, thioalkyl, substituted thioalkyl, hydroxy, halogen, amino, amido, carboxyl, —C(O)H, acyl, oxyacyl, carbamate, sulfonyl, sulfonamide, or sulfuryl; R⁴ is a 5-8 membered cycloalkyl optionally substituted with 1-8 R⁵ groups or a 5-8 membered cycloalkenyl optionally substituted with 1-8 R⁵ groups; each R⁵ can independently be: alkyl, substituted alkyl, alkenyl, substituted alkenyl, alkynyl, substituted alkynyl, aryl, substituted aryl, cycloalkyl, or substituted cycloalkyl; each of X¹, X², X³ and X⁴ can independently be O, NR⁶ and S; R⁶ is H or C₁-C₆ alkyl, and in which the therapeutically effective amount is sufficient to induce the expression of RKIP or PTEN, thereby inducing apoptosis.

In various aspects of the ninth embodiment, the expression of RKIP or PTEN is at least about 1, 2, 10, or 100 fold higher than in the absence of the compound of Formula I.

In a tenth embodiment, this invention provides a method of treating, preventing or inhibiting a cancer, the method comprising the step of administering to a subject a therapeutically effective amount of a compound of Formula I:

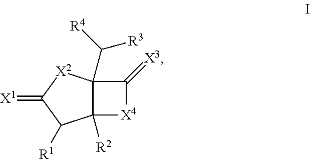

in which each of R¹, R² and R³ can independently be: H, alkyl, substituted alkyl, alkenyl, substituted alkenyl, alkynyl, substituted alkynyl, aryl, substituted aryl, heteroaryl, substituted heteroaryl, heterocyclic, substituted heterocyclic, cycloalkyl, substituted cycloalkyl, alkoxy, substituted alkoxy, thioalkyl, substituted thioalkyl, hydroxy, halogen, amino, amido, carboxyl, —C(O)H, acyl, oxyacyl, carbamate, sulfonyl, sulfonamide, or sulfuryl; R⁴ is a 5-8 membered cycloalkyl optionally substituted with 1-8 R⁵ groups or a 5-8 membered cycloalkenyl optionally substituted with 1-8 R⁵ groups; each R⁵ can independently be: alkyl, substituted alkyl, alkenyl, substituted alkenyl, alkynyl, substituted alkynyl, aryl, substituted aryl, cycloalkyl, or substituted cycloalkyl; each of X¹, X², X³ and X⁴ can independently be O, NR⁶ and S; R⁶ is H or C₁-C₆ alkyl, and in which the therapeutically effective amount is sufficient to inhibit the expression of YY1 or PTN, thereby inducing apoptosis or lowering the threshold of resistance to apoptosis by cytotoxic drugs.

In various aspects of the tenth embodiment, the expression of YY1 or PTN is at least about 1, 2, 10, or 100 fold lower than in the absence of the compound of Formula I.

In a eleventh embodiment, this invention provides a method of treating, preventing or inhibiting lymphoma by administering to a subject, optionally in combination with cytotoxic agents, a therapeutically effective amount of a compound of Formula I:

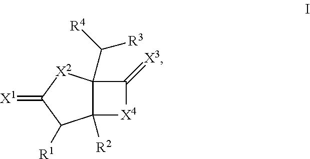

in which each of R¹, R² and R³ can be independently: H, alkyl, substituted alkyl, alkenyl, substituted alkenyl, alkynyl, substituted alkynyl, aryl, substituted aryl, heteroaryl, substituted heteroaryl, heterocyclic, substituted heterocyclic, cycloalkyl, substituted cycloalkyl, alkoxy, substituted alkoxy, thioalkyl, substituted thioalkyl, hydroxy, halogen, amino, amido, carboxyl, —C(O)H, acyl, oxyacyl, carbamate, sulfonyl, sulfonamide, or sulfuryl; R⁴ is a 5-8 membered cycloalkyl optionally substituted with 1-8 R⁵ groups or a 5-8 membered cycloalkenyl optionally substituted with 1-8 R⁵ groups; each R⁵ can independently be: alkyl, substituted alkyl, alkenyl, substituted alkenyl, alkynyl, substituted alkynyl, aryl, substituted aryl, cycloalkyl, or substituted cycloalkyl; each of X¹, X², X³ and X⁴ can independently be O, NR⁶ and S; R⁶ is H or C₁-C₆ alkyl, and in which the therapeutically effective amount is sufficient to induce the expression of RKIP or PTEN, thereby inducing apoptosis.

In a twelfth embodiment, this invention provides a method of treating, preventing or inhibiting lymphoma by administering to a subject a therapeutically effective amount of a compound of Formula I:

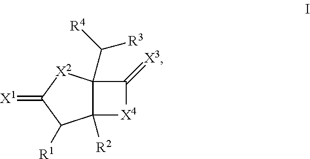

in which each of R¹, R² and R³ can be independently: H, alkyl, substituted alkyl, alkenyl, substituted alkenyl, alkynyl, substituted alkynyl, aryl, substituted aryl, heteroaryl, substituted heteroaryl, heterocyclic, substituted heterocyclic, cycloalkyl, substituted cycloalkyl, alkoxy, substituted alkoxy, thioalkyl, substituted thioalkyl, hydroxy, halogen, amino, amido, carboxyl, —C(O)H, acyl, oxyacyl, carbamate, sulfonyl, sulfonamide, or sulfuryl; R⁴ is a 5-8 membered cycloalkyl optionally substituted with 1-8 R⁵ groups or a 5-8 membered cycloalkenyl optionally substituted with 1-8 R⁵ groups; each R⁵ can independently be: alkyl, substituted alkyl, alkenyl, substituted alkenyl, alkynyl, substituted alkynyl, aryl, substituted aryl, cycloalkyl, or substituted cycloalkyl; each of X¹, X², X³ and X⁴ can independently be O, NR⁶ and S; R⁶ is H or C₁-C₆ alkyl, and in which the therapeutically effective amount is sufficient to inhibit the expression of YY1 or PTN, thereby inducing apoptosis.

In an thirteenth embodiment, this invention provides a method of treating, preventing or inhibiting a cancer with proteasome inhibitor therapy by administering to a subject a therapeutically effective amount of a compound of Formula I:

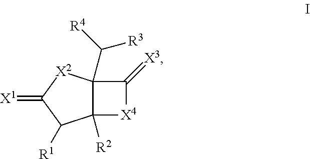

in which each of R¹, R² and R³ can independently be: H, alkyl, substituted alkyl, alkenyl, substituted alkenyl, alkynyl, substituted alkynyl, aryl, substituted aryl, heteroaryl, substituted heteroaryl, heterocyclic, substituted heterocyclic, cycloalkyl, substituted cycloalkyl, alkoxy, substituted alkoxy, thioalkyl, substituted thioalkyl, hydroxy, halogen, amino, amido, carboxyl, —C(O)H, acyl, oxyacyl, carbamate, sulfonyl, sulfonamide, or sulfuryl; R⁴ is a 5-8 membered cycloalkyl optionally substituted with 1-8 R⁵ groups or a 5-8 membered cycloalkenyl optionally substituted with 1-8 R⁵ groups; each R⁵ can independently be: alkyl, substituted alkyl, alkenyl, substituted alkenyl, alkynyl, substituted alkynyl, aryl, substituted aryl, cycloalkyl, or substituted cycloalkyl; each of X¹, X², X³ and X⁴ is independently selected from the group consisting of O, NR⁶ and S; and R⁶ is H or C₁-C₆ alkyl, in which the therapeutically effective amount is sufficient to induce the expression of RKIP or PTEN, thereby inducing apoptosis.

In an fourteenth embodiment, this invention provides a method of treating, preventing or inhibiting a cancer with proteasome inhibitor therapy by administering to a subject a therapeutically effective amount of a compound of Formula I:

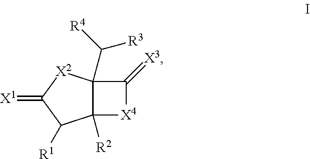

in which each of R¹, R² and R³ can independently be: H, alkyl, substituted alkyl, alkenyl, substituted alkenyl, alkynyl, substituted alkynyl, aryl, substituted aryl, heteroaryl, substituted heteroaryl, heterocyclic, substituted heterocyclic, cycloalkyl, substituted cycloalkyl, alkoxy, substituted alkoxy, thioalkyl, substituted thioalkyl, hydroxy, halogen, amino, amido, carboxyl, —C(O)H, acyl, oxyacyl, carbamate, sulfonyl, sulfonamide, or sulfuryl; R⁴ is a 5-8 membered cycloalkyl optionally substituted with 1-8 R⁵ groups or a 5-8 membered cycloalkenyl optionally substituted with 1-8 R⁵ groups; each R⁵ can independently be: alkyl, substituted alkyl, alkenyl, substituted alkenyl, alkynyl, substituted alkynyl, aryl, substituted aryl, cycloalkyl, or substituted cycloalkyl; each of X¹, X², X³ and X⁴ is independently selected from the group consisting of O, NR⁶ and S; and R⁶ is H or C₁-C₆ alkyl, in which the therapeutically effective amount is sufficient to induce the expression of YY1 or PTN thereby inducing apoptosis.

In a fifteenth embodiment of this invention, this invention provides a method of treating a therapy resistant cancer by administering to a subject a therapeutically effective amount of a compound of Formula I:

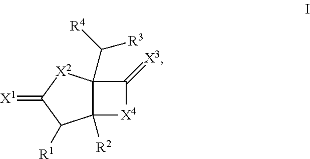

in which each of R¹, R² and R³ can independently be: H, alkyl, substituted alkyl, alkenyl, substituted alkenyl, alkynyl, substituted alkynyl, aryl, substituted aryl, heteroaryl, substituted heteroaryl, heterocyclic, substituted heterocyclic, cycloalkyl, substituted cycloalkyl, alkoxy, substituted alkoxy, thioalkyl, substituted thioalkyl, hydroxy, halogen, amino, amido, carboxyl, —C(O)H, acyl, oxyacyl, carbamate, sulfonyl, sulfonamide, or sulfuryl; R⁴ is a 5-8 membered cycloalkyl optionally substituted with 1-8 R⁵ groups or a 5-8 membered cycloalkenyl optionally substituted with 1-8 R⁵ groups; each R⁵ can independently be: alkyl, substituted alkyl, alkenyl, substituted alkenyl, alkynyl, substituted alkynyl, aryl, substituted aryl, cycloalkyl, or substituted cycloalkyl; each of X¹, X², X³ and X⁴ can independently be O, NR⁶ and S; R⁶ is H or C₁-C₆ alkyl, in which the therapeutically effective amount is sufficient to induce the expression of RKIP or PTEN, thereby inducing apoptosis.

In a sixteenth embodiment of this invention, this invention provides a method of treating a therapy resistant cancer by administering to a subject a therapeutically effective amount of a compound of Formula I:

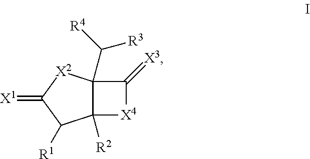

in which each of R¹, R² and R³ can independently be: H, alkyl, substituted alkyl, alkenyl, substituted alkenyl, alkynyl, substituted alkynyl, aryl, substituted aryl, heteroaryl, substituted heteroaryl, heterocyclic, substituted heterocyclic, cycloalkyl, substituted cycloalkyl, alkoxy, substituted alkoxy, thioalkyl, substituted thioalkyl, hydroxy, halogen, amino, amido, carboxyl, —C(O)H, acyl, oxyacyl, carbamate, sulfonyl, sulfonamide, or sulfuryl; R⁴ is a 5-8 membered cycloalkyl optionally substituted with 1-8 R⁵ groups or a 5-8 membered cycloalkenyl optionally substituted with 1-8 R⁵ groups; each R⁵ can independently be: alkyl, substituted alkyl, alkenyl, substituted alkenyl, alkynyl, substituted alkynyl, aryl, substituted aryl, cycloalkyl, or substituted cycloalkyl; each of X¹, X², X³ and X⁴ can independently be O, NR⁶ and S; R⁶ is H or C₁-C₆ alkyl, in which the therapeutically effective amount is sufficient to inhibit the expression of YY1 or PTN, thereby inducing apoptosis.

### BRIEF DESCRIPTION OF THE DRAWINGS

FIG. 1 shows that Salinosporamide A significantly sensitizes B-NHL Ramos cell line to CDDP-induced apoptosis.

Drug and Salinosporamide A resistanct Ramos cells (10⁶ ml) were treated with various concentrations of Salinosporamide A for one hour and then treated with CDDP (15 μg/ml) for an additional 20 h. The cultures were then washed and the cells harvested and examined for apoptosis using the propidium iodide method, which measures DNA fragmentation, by flow cytometry. The percent apoptotic cells was recorded. Very long concentrations of Salinosporamide A (0.1 nM) were effective. The treatment were performed in duplicates.

FIG. 2A shows that Salinosporamide A sensitizes drug and Salinosporamide A resistant B-NHL Daudi cell line to CDDP-induced apoptosis. Daudi cells were treated for Ramos as in FIG. 1 above.

FIG. 2B shows that Salinosporamide A sensitizes B-NHL Rituximab resistant Daudi RR1 cell line to CDDP-induced apoptosis. The rituximab resistant clone Daudi-RR1 was treated as in FIG. 1 above.

FIG. 3A compares Salinosporamide A and bortezomib-induced sensitization of B-NHL Daudi WT cells to CDDP-induced apoptosis. Daudi cells were treated with various concentrations of bortezomib or Salinosporamide A for 1 h and then treated with CDDP (10 μg/ml) for an additional 20 h and the cells were treated for apoptosis as described in FIG. 1 above.

FIG. 3B compares Salinosporamide A and DHMEQ-induced sensitization of B-NHL Daudi WT cells to CDDP-induced apoptosis. Daudi cells were treated with various concentrations of DHMEQ (μM) and/or Salinosporamide A (nM) for 1 h and then treated with CDDP (10 μg/ml) for an additional 20 h. The cells were then harvested and tested for apoptosis as described in FIG. 1 above.

FIG. 4A compares the effect of Salinosporamide A and bortezomib on CDDP-induced apoptosis in Daudi RR1 cells. The rituximab resistant Daudi RR1 clone was treated with various concentrations of bortezomib and/or Salinosporamide A for 1 h and then treated with CDDP 10 μg/ml for an additional 20 h. These cells were then examined for apoptosis as described in FIG. 1 above.

FIG. 4B compares the effect of Salinosporamide A and DHMEQ on CDDP-induced apoptosis in Daudi RR1 cells. The rituximab resistant Daudi RR1 cells were treated with various concentrations of DHMEQ (μM) or Salinosporamide A (nM) for 1 h and then treated with CDDP (10 μg/ml) for an additional 20 h. The cells were then examined for apoptosis as described in FIG. 1 above.

FIG. 5 shows rituximab-mediated sensitization to Salinosporamide A-induced apoptosis. Ramos cells were treated with rituximab (20 μg/ml) for 1 h and then treated with various concentrations of Salinosporamide A for an additional 20 h. The cells were then examined for apoptosis as described above in FIG. 1.

FIG. 6 shows that in comparison to rituximab-mediated chemosensitization to CDDP, rituximab sensitizes to Salinosporamide A-induced apoptosis to a higher level than CDDP.

FIG. 7 shows the structure of Salinosporamide A.

FIG. 8 shows that rituximab sensitizes Ramos B-NHL cells to Salinosporamide A-induced apoptosis. Ramos cells were cultured untreated or treated with rituximab (20 μg/ml) for 18 h. Thereafter, the cells were treated with adriamycin (ADR) (5 μg/ml) or with different concentrations of Salinosporamide A (0.1, 1.0, 10 nM) overnight. The cell lines were examined for apoptosis by flow cytometry using the propidium iodide method for measuring DNA fragmentation.

FIG. 9 shows that treatment of tumor cells with Salinosporamide A results in the induction of the tumor suppressor, Raf-kinase inhibitor protein (RKIP). Ramos cells were treated with 10 nM Salinosporamide A for various periods of time, and total cell lysates were examined for the expression of RKIP by western blot analysis. The expression of β-actin was used as a control.

FIG. 10 shows that treatment of tumor cells with Salinosporamide A inhibits YY1 expression in Ramos B-NHL cells. Ramos cells were treated with various concentrations of Salinosporamide A for 24 hours, and total cell lysates were examined for the expression of YY1 by western blot analysis. The expression of β-actin was used as a control.

FIG. 11 shows a schematic diagram representing the effect of Salinosporamide A on sensitization of drug-resistant tumor cells to various drug-immune-induced apoptosis. Tumor cells constitutively express activated NF-κB, which in turn regulates the transcription of various survival genes and anti-apoptotic genes as well as regulating the expression of the transcriptional repressor YY1. Treatment of the cells with Salinosporamide A results in inhibition of NF-κB activity leading to inhibition of survival gene products and anti-apoptotic gene products and resulting in chemosensitization. In addition, inhibition of NF-κB by Salinosporamide A also inhibits YY1, which we have shown to negatively regulate Fas and DR5 transcription. The inhibition of YY1 results in upregulation of Fas and DR5 expression and sensitizes cells to FasL and TRAIL-induced apoptosis.

FIG. 12 shows a schematic diagram representing tumor cells that express constitutively activated NF-κB, which regulates several anti-apoptotic gene products such as Bcl-2, Bcl-xL, and Mcl-1. Previous studies have demonstrated that those anti-apoptotic gene products are important for maintaining tumor cell resistance to various chemotherapeutic drugs. We have shown that treatment with rituximab inhibits NF-κB activity and also inhibits the above anti-apoptotic gene products and sensitizes the tumor cells to various chemosterapeutic drugs. Salinosporamide A also inhibits NF-κB activity, and the combination of rituximab and Salinosporamide A results in complementation or synergy and apoptosis. The apoptosis is the result of rituximab-induced sensitization to Salinosporamide A apoptosis.

FIG. 13 demonstrates that Salinosporamide A activates caspase 9 in Ramos cells and in combination with CDDP more caspase 9 was activated. The activation of capsase 9 was assessed by western as the level of pro-caspase 9 was reduced following treatment.

FIG. 14 shows that Salinosporamide A inhibits the anti-apoptotic gene product Bcl-xL following treatment with very low concentrations (<2.5 nM). Bcl-xL expression was assessed by western.

FIG. 15 shows that Salinosporamide A induces the expression of RKIP and PTEN and in combination with rituximab more was expressed as assessed by western.

FIG. 16 shows that Salinosporamide A inhibits the growth factor pleiotrophin (PTN) expression significantly at the concentration of 5 nM.

FIG. 17A shows that Salinosporamide A treatment of Ramos upregulated DR5 surface expression as detected by flow cytometry.

FIG. 17B shows that Salinosporamide A upregulates DR5 expression in the rituximab resistant Ramos cells RR1.

FIG. 18 shows that Salinosporamide A sensitizes TRAIL-resistant Ramos cells to TRAIL-induced apoptosis.

FIG. 19 shows that Salinosporamide A upregulates DR5 expression in Ramos cells as determined by Western.

FIG. 20 shows that Salinosporamide A inhibits YY1 transcription as determined by RT-PRC.

### DETAILED DESCRIPTION OF THE INVENTION

### I. Definitions

“Cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas) and Hodgkin's lymphoma, leukemia (including AML, ALL, and CML), multiple myeloma, mantle cell lymphoma, Waldenstrom's macrogobulinemia, and Philadelphia positive cancers.

“Therapy resistant” cancers, tumor cells, and tumors refers to cancers that have become resistant to both apoptosis-mediated (e.g., through death receptor cell signaling, for example, Fas ligand receptor, TRAIL receptors, TNF-R1), various conventionally used chemotherapeutic drugs, hormonal drugs, and radiation, and non-apoptosis mediated (e.g., antimetabolites, anti-angiogenic, etc.) cancer therapies. “Therapy sensitive” cancers are not resistant to therapy. One of skill in the art will appreciate that some cancers are therapy sensitive to particular agents but not to others. Cancer therapies include chemotherapy, hormonal therapy, radiotherapy, immunotherapy, and gene therapy.

“Therapy-mediated or induced cytotoxicity” refers to all mechanisms by which cancer therapies kill or inhibit cancer cells, including but not limited to inhibition of proliferation, inhibition of angiogenesis, and cell death due to, for example, activation of apoptosis pathways (e.g., death receptor cell signaling, for example, Fas ligand receptor, TRAIL receptors, TNF-R1). Cancer therapies include chemotherapy, immunotherapy, radiotherapy, and hormonal therapy.

“Therapeutic treatment” and “cancer therapies” and “cancer therapy reagents” refers to apoptosis-mediated and non-apoptosis mediated cancer therapies that treat, prevent, or inhibit cancers, including chemotherapy, hormonal therapy (e.g., androgens, estrogens, antiestrogens (tamoxifen), progestins, thyroid hormones and adrenal cortical compounds), radiotherapy, and immunotherapy (e.g., ZEVALIN, BEXXAR, RITUXAN (rituximab), HERCEPTIN). Cancer therapies can be enhanced by administration with a sensitizing agent, as described herein, either before or with the cancer therapy.

“Chemotherapeutic drugs” include conventional chemotherapeutic reagents such as alkylating agents, anti-metabolites, plant alkaloids, antibiotics, and miscellaneous compounds e.g., cis-platinum, CDDP, methotrexate, vincristine, adriamycin, bleomycin, and hydroxyurea. Chemotherapeutic drugs also include proteasome inhibitors such as salinosporamides (e.g., Salinosporamide A), bortezomib, PS-519, omuralide, PR-171 and its analogs, and Gleevec. The drugs can be administered alone or combination (“combination chemotherapy”).

By “sensitizingly effective amount or dose” or “sensitizingly sufficient amount or dose” herein is meant a dose that produces cancer cell sensitizing effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). Sensitized cancer cells respond better to cancer therapy (are inhibited or killed faster or more often) than non-sensitized cells, as follows: Control samples (untreated with sensitizing agents) are assigned a relative cancer therapy response value of 100%. Sensitization is achieved when the cancer therapy response value relative to the control is about 110% or 120%, preferably 200%, more preferably 500-1000% or more, i.e., at least about 10% more cells are killed or inhibited, or the cells are killed or inhibited at least about 10% faster. Cancer therapy response value refers to the amount of killing or inhibition of a cancer cell, or the speed of killing or inhibition of a cancer cell when it is treated with a cancer therapy. Some compounds are useful both as therapeutic reagents and as sensitizing reagents. Often, a lower dose (i.e., lower than the conventional therapeutic dose) or sub-toxic dose of such a reagent can be used to sensitize a cell. Often, when a cell is sensitized, a lower dose of the chemotherapeutic reagent can be used to achieve the same therapeutic effect as with a cell that has not been sensitized.

By “therapeutically effective amount or dose” or “therapeutically sufficient amount or dose” herein is meant a dose that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). In sensitized cells, the therapeutically effective dose can often be lower than the conventional therapeutically effective dose for non-sensitized cells.

Apoptosis refers to a process of programmed cell death that is different from the general cell death or necrosis that results from exposure of cells to non-specific toxic events such as metabolic poisons or ischemia in being an ordered molecular process by which unwanted cells undergo death. Cells undergoing apoptosis show characteristic morphological changes such as chromatin condensation and fragmentation and breakdown of the nuclear envelope in a process called pyknosis. As apoptosis proceeds, the plasma membrane is seen to form blebbings cells and the apoptotic cells are either phagocytosed or else break up into smaller vesicles which are then phagocytosed. Typical assays used to detect and measure apoptosis include microscopic examination of pyknotic bodies as well as enzymatic assays such as TUNEL labeling, caspase assay, annexin assay, and DNA laddering, among others. Apoptotic cells can be quantitated by FACS analysis of cells stained with propidium iodide for DNA hypoploidy.

“Inducing apoptosis” refers to an agent or process which causes a cell to undergo the program of cell death described above for apoptosis.

“Salinosporamide” refers to proteasome inhibitor compounds produced by Salinospora sp., a marine gram positive actinomycete, e.g., Salinosporamide A (Salinosporamide A), B, C, etc, and analogs thereof. Salinosporamides can be made by isolating the products from fermentation of Salinospora (wild type and mutant strains) and genetically engineered microorganisms, by biosynthesis in vitro using whole cells, enzymes, and recombinant enzymes, and by synthetic chemistry techniques.

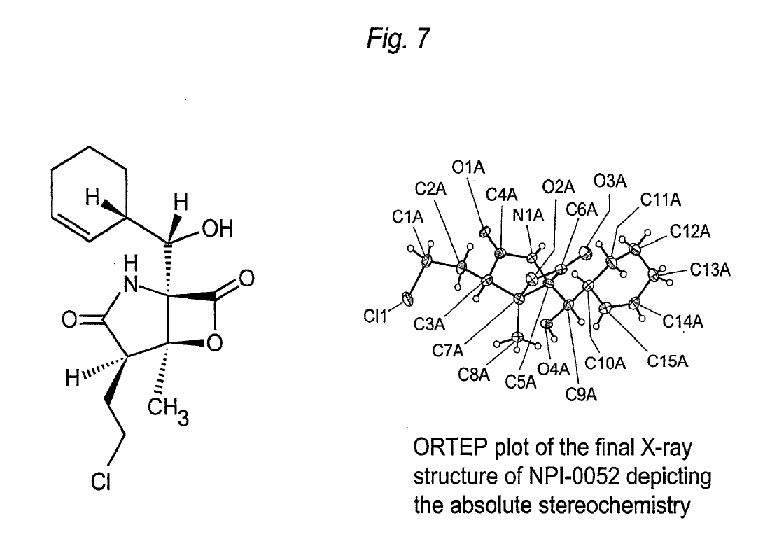

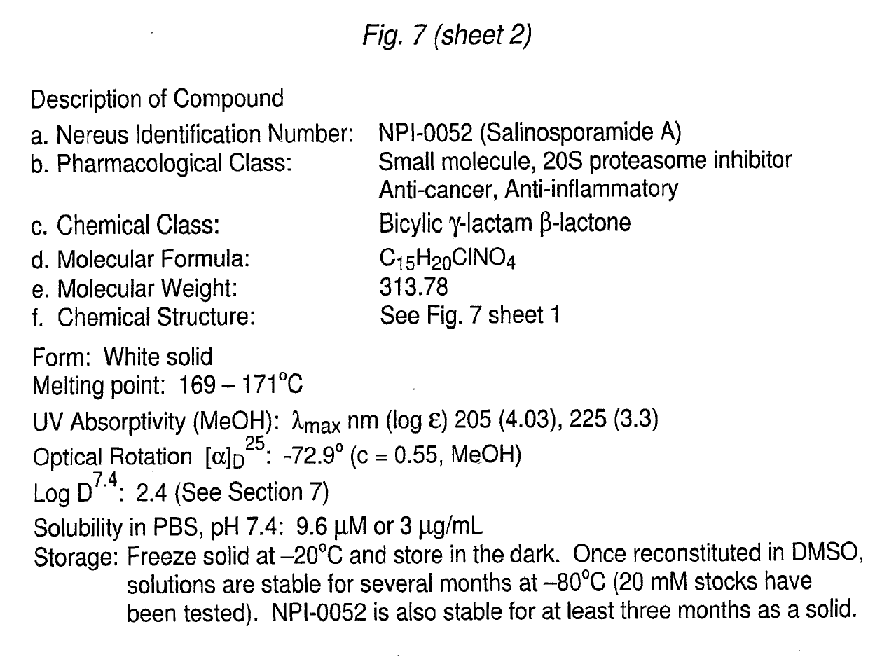

“Salinosporamide A” refers to proteasome inhibitor compounds produced by Salinospora sp., a marine gram positive actinomycete. This term also refers to analogs of Salinosporamide A. Salinosporamide A and analogs thereof have structures as disclosed herein, e.g., in Formula 1 and FIG. 7, as well as in US20050049294, herein incorporated by reference.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990))

For preparation of antibodies, e.g., recombinant, monoclonal, or polyclonal antibodies, many technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (U.S. Pat. No. 4,946,778, U.S. Pat. No. 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

As used herein, the term “alkyl” refers to a monovalent straight or branched chain hydrocarbon group having from one to about 12 carbon atoms, including methyl, ethyl, n-propyl, isopropyl, n-butyl, isobutyl, tert-butyl, pentyl, hexyl, and the like.

As used herein, the term “substituted alkyl” refers to alkyl groups further bearing one or more substituents selected from hydroxy, alkoxy, mercapto, cycloalkyl, substituted cycloalkyl, heterocyclic, substituted heterocyclic, aryl, substituted aryl, heteroaryl, substituted heteroaryl, aryloxy, substituted aryloxy, halogen, cyano, nitro, amino, amido, —C(O)H, acyl, oxyacyl, carboxyl, sulfonyl, sulfonamide, sulfuryl, and the like.

As used herein, the term “lower alkyl” refers to alkyl groups having from 1 to about 6 carbon atoms.

As used herein, the term “alkenyl” refers to straight or branched chain hydrocarbyl groups having one or more carbon-carbon double bonds, and having in the range of about 2 up to 12 carbon atoms, and “substituted alkenyl” refers to alkenyl groups further bearing one or more substituents as set forth above. Alkenyl groups useful in the present invention include, but are not limited to, ethenyl, n-propenyl, isopropenyl, n-butenyl, isobutenyl, pentenyl, hexenyl, and the like.

As used herein, the term “alkynyl” refers to straight or branched chain hydrocarbyl groups having at least one carbon-carbon triple bond, and having in the range of about 2 up to 12 carbon atoms, and “substituted alkynyl” refers to alkynyl groups further bearing one or more substituents as set forth above. Alkynyl groups useful in the present invention include, but are not limited to, ethynyl, propynyl, butyryl, pentynyl, hexynyl, and the like.

As used herein, the term “aryl” refers to aromatic groups having in the range of 6 up to 14 carbon atoms and 1 to 3 rings, and “substituted aryl” refers to aryl groups further bearing one or more substituents as set forth above. Aryl groups useful in the present invention include, but are not limited to, phenyl, benzyl, naphthyl, biphenyl, phenanthrenyl, and anthrenyl.

As used herein, the term “heteroaryl” refers to aromatic rings containing one or more heteroatoms (e.g., N, O, S, or the like) as part of the ring structure, having in the range of 3 up to 14 carbon atoms and 1 to 3 rings. “Substituted heteroaryl” refers to heteroaryl groups further bearing one or more substituents as set forth above. Heteroaryl groups useful in the present invention include, but are not limited to, pyridyl, pyridyl N-oxide, indolyl, indazolyl, quinoxalinyl, quinolinyl, isoquinolinyl, benzothienyl, benzopyranyl, benzopyranyl, benzothiopyranyl, furanyl, pyrrolyl, thiazolyl, benzothiazolyl, oxazolyl, isoxazolyl, triazolyl, tetrazolyl, pyrazolyl, imidazolyl, and thienyl.

As used herein, the term “alkoxy” refers to the moiety —O-alkyl-, wherein alkyl is as defined above, and “substituted alkoxy” refers to alkoxyl groups further bearing one or more substituents as set forth above.

As used herein, the term “thioalkyl” refers to the moiety —S-alkyl-, wherein alkyl is as defined above, and “substituted thioalkyl” refers to thioalkyl groups further bearing one or more substituents as set forth above.

As used herein, the term “cycloalkyl” refers to ring-containing alkyl groups containing in the range of about 3 up to 8 carbon atoms, and “substituted cycloalkyl” refers to cycloalkyl groups further bearing one or more substituents as set forth above. Cycloalkyl groups useful in the present invention include, but are not limited to, cyclopropane, cyclobutane, cyclopentane, cyclohexane, cycloheptane and cyclooctane.

As used herein, the term “cycloalkenyl” refers to a 3 to 8 membered cycloalkyl group having at least one carbon-carbon double bond (alkene) in the ring, and “substituted cycloalkenyl” refers to cycloalkenyl groups further bearing one or more substituents as set forth above. Cycloalkenyl rings useful in the present invention include, but are not limited to, 1-cyclopentenyl, 2-cyclopentenyl, 3-cyclopentenyl, 1-cyclohexenyl, 2-cyclohexenyl, 3-cyclohexenyl, as well as cyclopropenyl, cyclobutenyl, cycloheptenyl and cyclooctenyl. Cycloalkadienyls are also useful in the present invention and include, but are not limited to, cyclopentadienyl, cyclohexadienyl, cycloheptadienyl and cyclooctadienyl.

As used herein, the term “heterocyclic”, refers to cyclic (i.e., ring-containing) groups containing one or more heteroatoms (e.g., N, O, S, or the like) as part of the ring structure, having in the range of 3 up to 14 carbon atoms and 1 to 3 rings. “Substituted heterocyclic” refers to heterocyclic groups further bearing one or more substituents as set forth above. Heterocyclic groups useful in the present invention, include, but are not limited to, pyrrolidinyl, pyrrolinyl, imidazolinyl, pyrazolidinyl, pyrazolinyl, piperidinyl, piperazinyl, indolinyl, quinuclidinyl, morpholinyl, tetrahydrofuranyl, tetrahydrothienyl and dioxane.

The compounds of the invention may be formulated into pharmaceutical compositions as natural or salt forms. Pharmaceutically acceptable non-toxic salts include the base addition salts (formed with free carboxyl or other anionic groups) which may be derived from inorganic bases such as, for example, sodium, potassium, ammonium, calcium, or ferric hydroxides, and such organic bases as isopropylamine, trimethylamine, 2-ethylamino-ethanol, histidine, procaine, and the like. Such salts may also be formed as acid addition salts with any free cationic groups and will generally be formed with inorganic acids such as, for example, hydrochloric, sulfuric, or phosphoric acids, or organic acids such as acetic, p-toluenesulfonic, methanesulfonic acid, oxalic, tartaric, mandelic, and the like. Salts of the invention include amine salts formed by the protonation of an amino group with inorganic acids such as hydrochloric acid, hydrobromic acid, hydroiodic acid, sulfuric acid, phosphoric acid, and the like. Salts of the invention also include amine salts formed by the protonation of an amino group with suitable organic acids, such as p-toluenesulfonic acid, acetic acid, and the like. Additional excipients which are contemplated for use in the practice of the present invention are those available to those of ordinary skill in the art, for example, those found in the United States Pharmacopeia Vol. XXII and National Formulary Vol. XVII, U.S. Pharmacopeia Convention, Inc., Rockville, Md. (1989), the relevant contents of which is incorporated herein by reference.

The compounds according to this invention may contain one or more asymmetric carbon atoms and thus occur as racemates and racemic mixtures, single enantiomers, diastereomeric mixtures and individual diastereomers. The term “stereoisomer” refers to chemical compounds which differ from each other only in the way that the different groups in the molecules are oriented in space. Stereoisomers have the same molecular weight, chemical composition, and constitution as another, but with the atoms grouped differently. That is, certain identical chemical moieties are at different orientations in space and, therefore, when pure, have the ability to rotate the plane of polarized light. However, some pure stereoisomers may have an optical rotation that is so slight that it is undetectable with present instrumentation. All such isomeric forms of these compounds are expressly included in the present invention.

Each stereogenic carbon may be of R or S configuration. Although the specific compounds exemplified in this application may be depicted in a particular configuration, compounds having either the opposite stereochemistry at any given chiral center or mixtures thereof are also envisioned. When chiral centers are found in the derivatives of this invention, it is to be understood that this invention encompasses all possible stereoisomers. The terms “optically pure compound” or “optically pure isomer” refers to a single stereoisomer of a chiral compound regardless of the configuration of the compound.

### II. Compounds

Compounds useful in the present invention include those of Formula I:

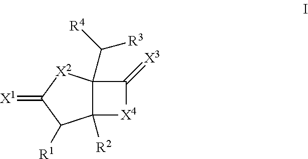

wherein each of R¹, R² and R³ are independently selected from the group consisting of H, alkyl, substituted alkyl, alkenyl, substituted alkenyl, alkynyl, substituted alkynyl, aryl, substituted aryl, heteroaryl, substituted heteroaryl, heterocyclic, substituted heterocyclic, cycloalkyl, substituted cycloalkyl, alkoxy, substituted alkoxy, thioalkyl, substituted thioalkyl, hydroxy, halogen, amino, amido, carboxyl, —C(O)H, acyl, oxyacyl, carbamate, sulfonyl, sulfonamide, and sulfuryl. R⁴ is a 5-8 membered cycloalkyl optionally substituted with 1-8 R⁵ groups or a 5-8 membered cycloalkenyl optionally substituted with 1-8 R⁵ groups. Each R⁵ is independently selected from the group consisting of alkyl, substituted alkyl, alkenyl, substituted alkenyl, alkynyl, substituted alkynyl, aryl, substituted aryl, cycloalkyl, and substituted cycloalkyl. Each of X¹, X², X³ and X⁴ is independently selected from the group consisting of O, NR⁶ and S. And R⁶ is H or C₁-C₆ alkyl.

Additional compounds useful in the present invention include the following:

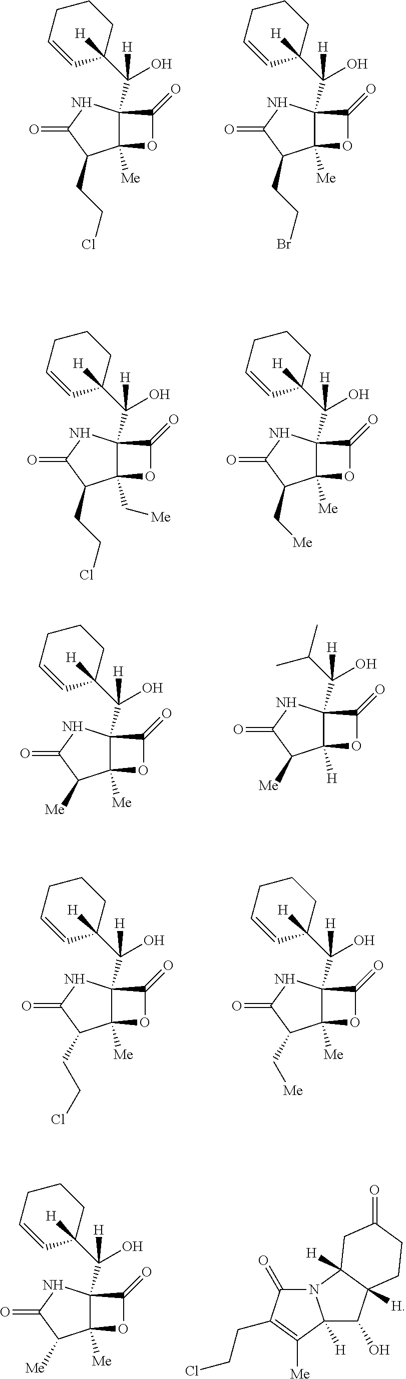

Salinosporamides disclosed in J. Org. Chem., 70(16), 6196-6203, 2005 are incorporated herein by reference. Additional Salinsoporamides are described in US20050049294, herein incorporated by reference in its entirety.

The compounds of the present invention can be prepared by a variety of methods including fermentation, recombinant biosynthesis and via synthetic methodologies.

A. Fermentation

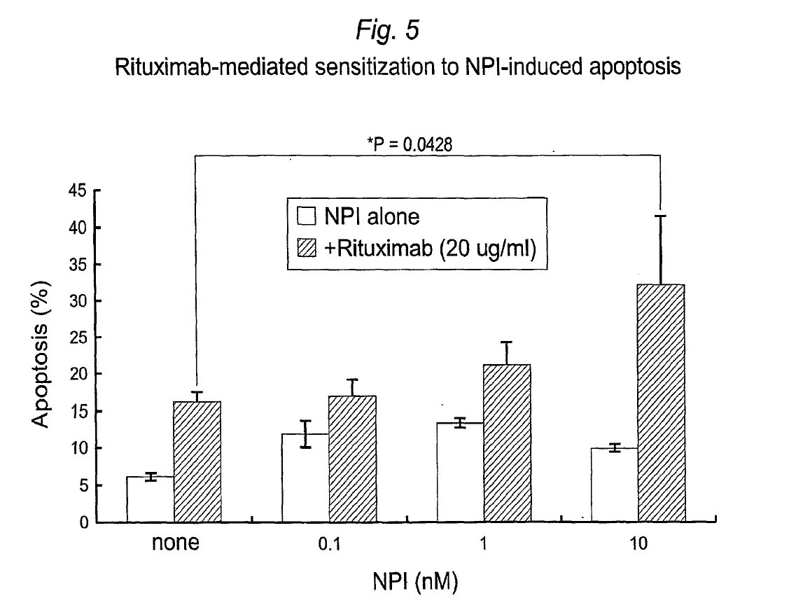

The compounds of the present invention can be prepared, for example, by bacterial fermentation, which generates the compounds in sufficient amounts for pharmaceutical drug development and for clinical trials. In some embodiments, invention compounds are produced by fermentation of the actinomycete strains CNB392 and CNB476 in A1Bfe+C or CKA-liquid media. Essential trace elements which are necessary for the growth and development of the culture should also be included in the culture medium. Such trace elements commonly occur as impurities in other constituents of the medium in amounts sufficient to meet the growth requirements of the organisms. It may be desirable to add small amounts (i.e. 0.2 mL/L) of an antifoam agent such as polypropylene glycol (M.W. about 2000) to large scale cultivation media if foaming becomes a problem. The organic metabolites are isolated by adsorption onto an amberlite XAD-16 resin. For example, Salinosporamide A is isolated by elution of the XAD-16 resin with methanol:dichlormethane 1:1, which affords about 105 mg crude extract per liter of culture. Salinosporamide A is then isolated from the crude extract by reversed-phase flash chromatography followed by reverse-phase HPLC and normal phase HPLC, which yields 6.7 mg of Salinosporamide A. FIG. 5 and Example 1 of US 2004/0259856 (incorporated herein by reference) set forth a fermentation procedure for the preparation of the compounds of the instant invention. US20050049294, herein incorporated by reference in its entirety, also provides methods of isolating the compounds from fermentation broth.

B. Recombinant Biosynthesis

Recombinant biosynthesis uses cells expressing cloned genes and optionally naturally occurring pathways to create biosynthetic pathways to produce natural and novel metabolites (see, e.g., Altreuter et al., Curr. Opin. Biotehcnol. 10:130-136 (1999); Reynolds, PNAS 95:12744-12746 (1998); and Cane et al., Science 282:63-68 (1998)). Several biosynthetic pathways are possible for the production of the compounds of the present invention, including a mixed polyketide-non-ribosomal peptide synthesis pathway. Polyketides and non-ribosomal peptides are synthesized from small chain carboxylic acid and amino acid monomers, respectively, by large multifunctional protein complexes called polyketide synthetases and nonribosomal peptide synthetases. US20050049294, herein incorporated by reference in its entirety, also provides information on recombinant biosynthesis.

C. Synthetic Procedure

The compounds of the present invention can also be prepared using standard organic synthesis procedures known in the art. An exemplary synthetic procedure can be found in US 2005/0228186 (incorporated herein by reference) for the synthesis of

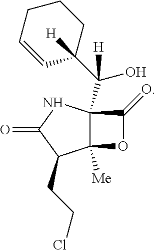

One of skill in the art will recognize that additional pathways exist for the synthetic preparation of the compounds of the present invention. US20050049294, herein incorporated by reference in its entirety, also provides information on synthesis of the compounds.

### III. Methods

As described herein, Salinosporamide A is useful for sensitizing both sensitive and resistant cancer cells to therapy based apoptosis when administered in combination with low dose or sub-toxic amounts of cancer therapeutic reagents. Salinosporamide A and the low dose or sub-toxic amount of a cancer therapeutic can be administered alone to sensitize cells for subsequent therapies or co-administered in combination with chemotherapy, radiotherapy, hormonal therapy, or immunotherapy. In another embodiment, Salinosporamide A is used as a chemotherapeutic agent after cellular sensitization using an antibody. Salinosporamide A as a therapeutic can be administered alone or co-administered in combination with chemotherapy, radiotherapy, hormonal therapy, or immunotherapy. Methods of using Salinosporamide A are also described in US patent application 20050239866 and 20050049294, herein incorporated by reference in their entirety.

Pharmaceutically acceptable carriers are determined in part by the particular composition being administered, as well as by the particular method used to administer the composition. Accordingly, there are a wide variety of suitable formulations of pharmaceutical compositions of the present invention (see, e.g., Remington's Pharmaceutical Sciences, 20^{th} ed., 2003, supra).

Formulations suitable for oral administration can consist of (a) liquid solutions, such as an effective amount of the compound suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The compound of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Suitable formulations for rectal administration include, for example, suppositories, which consist of the compound with a suppository base. Suitable suppository bases include natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, and intravenous administration are the preferred methods of administration. The formulations of compounds can be presented in unit-dose or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents.

Preferred pharmaceutical preparations deliver one or the compounds of the invention, optionally in combination with one or more therapeutic agents, in a sustained release formulation. Typically, Salinosporamide A is administered therapeutically as a sensitizing agent that increases the susceptibility of tumor cells to other cytotoxic cancer therapies, including chemotherapy, radiation therapy, immunotherapy and hormonal therapy. In some embodiments, Salinosporamide A acts as a chemotherapeutic reagent after cellular sensitization using an antibody.

In therapeutic use for the treatment of cancer, the compounds utilized in the pharmaceutical method of the invention are administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound being employed. For example, dosages can be empirically determined considering the type and stage of cancer diagnosed in a particular patient. The dose administered to a patient, in the context of the present invention should be sufficient to effect a beneficial therapeutic response in the patient over time. The size of the dose also will be determined by the existence, nature, and extent of any adverse side-effects that accompany the administration of a particular compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

The pharmaceutical preparations are typically delivered to a mammal, including humans and non-human mammals. Non-human mammals treated using the present methods include domesticated animals (i.e., canine, feline, murine, rodentia, and lagomorpha) and agricultural animals (bovine, equine, ovine, porcine).

### EXAMPLES

The following examples are offered to illustrate, but not to limit the claimed invention.

### Example I

### Salinosporamide a Induced Sensitization

### 1) Salinosporamide A-Induced Sensitization of Drug-Resistant B-NHL Ramos and Daudi Cell Lines to CDDP-Induced Apoptosis

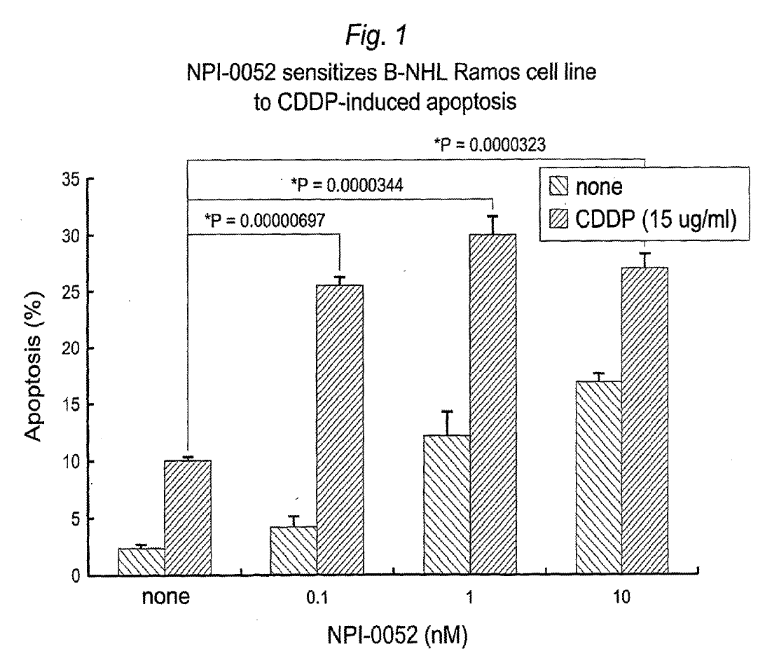

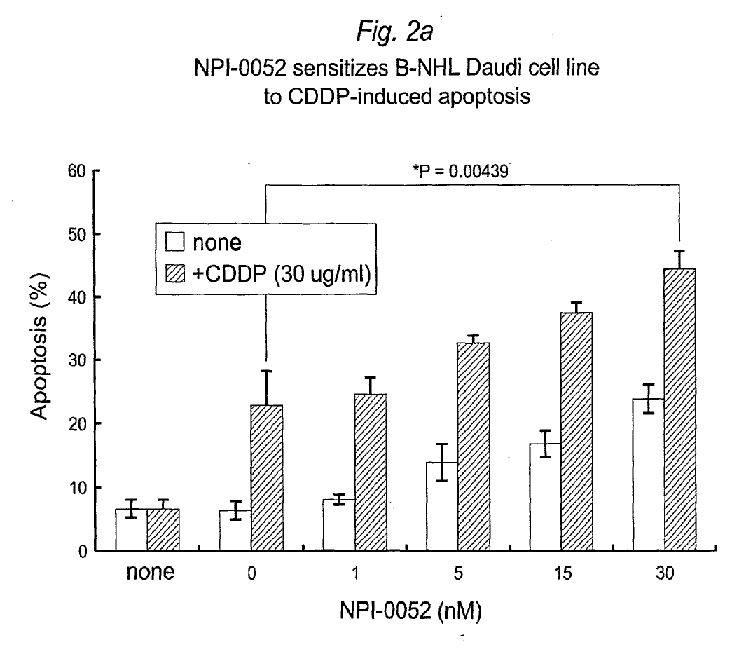

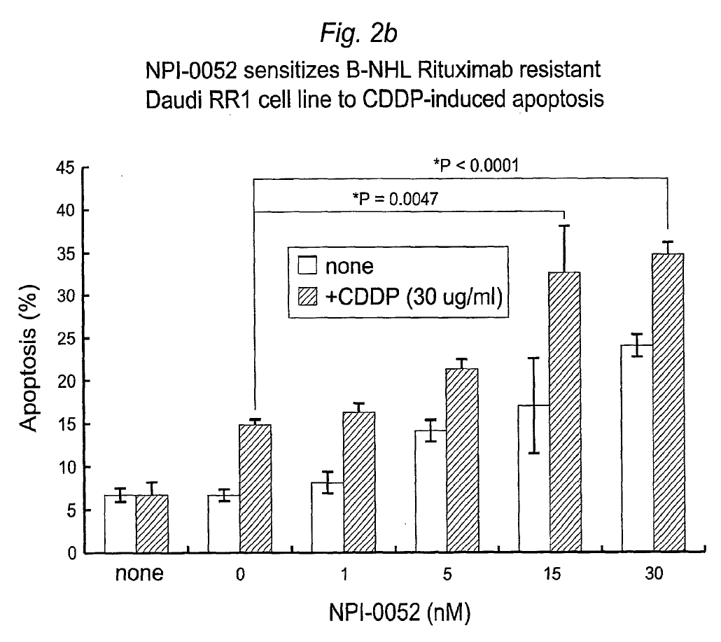

The CDDP resistant B-NHL Ramos cell line was treated with various concentrations of Salinosporamide A for one hour and then treated with predetermined nontoxic concentration of CDDP (15 μg/ml) for an additional 20 hours. The cells were then harvested and examined for apoptosis using the propidium iodide (PI) technique by flow cytometry examining DNA fragmentation. FIG. 1 shows that the combination treatment with Salinosporamide A and CDDP resulted in significant potentiation of cytotoxicity. In addition, Salinosporamide A treatment alone showed modest cytotoxicity at the concentration of 1 and 10 nM. The potentiation of cytotoxicity was mostly observed at very low concentrations of Salinosporamide A (0.1 nM) and significant synergistic cytotoxicity was observed. Similar studies were performed with the Daudi B-NHL cell line. Like Ramos, significant cytotoxicity was observed and the extent of cytotoxicity was a function of the concentration of Salinosporamide A used (FIG. 2A). These findings demonstrate that Salinosporamide A sensitizes both Ramos and Daudi B-NHL cells to CDDP-induced apoptosis.

### 2) Salinosporamide A-Mediated Sensitization of Rituximab Resistant Daudi Clone (Daudi RR1) to CDDP-Induced Apoptosis.

Rituximab (chimeric anti-CD20 monoclonal antibody) has been used in the treatment of Non-Hodgkin's Lymphoma alone or in combination with chemotherapy. The clinical response has been very encouraging; however, some patients are initially unresponsive or develop resistance following treatment. In order to investigate the mechanism of rituximab resistance we have developed in our laboratory rituximab resistant clones from B-NHL cell lines. We have selected certain clones for further analysis of the underlying mechanism of resistance. In the present study we have examined the Daudi RR1 clone which is resistant to rituximab-induced signaling and unlike Daudi wild type, rituximab failed to sensitize Daudi RR1 to drug-induced apoptosis. In addition, we have found that Daudi RR1 also develops the highest degree of drug resistance compared to wild type. We investigated whether Salinosporamide A can sensitize Daudi RR1 to CDDP-induced apoptosis. FIG. 2B demonstrates that indeed Salinosporamide A significantly sensitized Daudi RR1 to CDDP-induced apoptosis and the extent of potentiation of cytotoxicity was a function of the concentration of Salinosporamide A used. These findings demonstrate that Salinosporamide A may be used clinically to reverse rituximab resistance to chemotherapy.

3) Comparison Between Salinosporamide A and Bortezomib in their Ability to Sensitize B-NHL Cell Lines to CDDP-Induced Apoptosis

a) Study with Daudi and Wild Type Cells

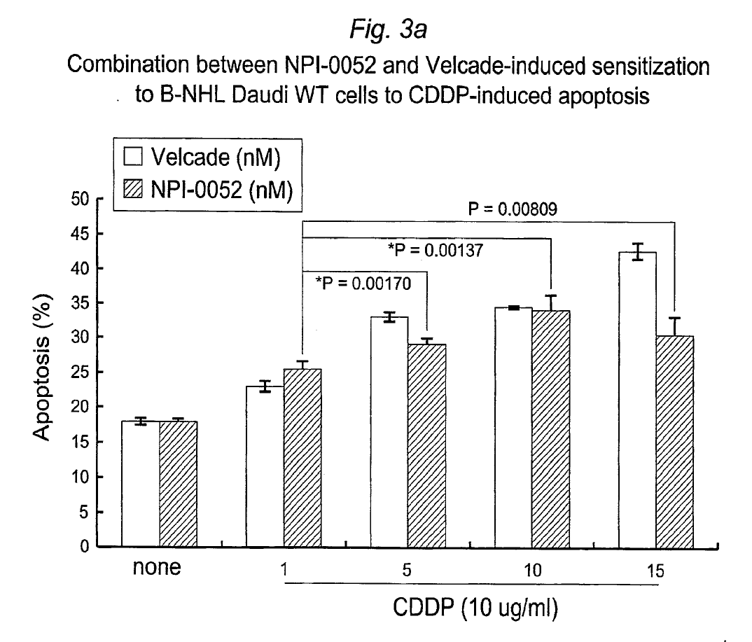

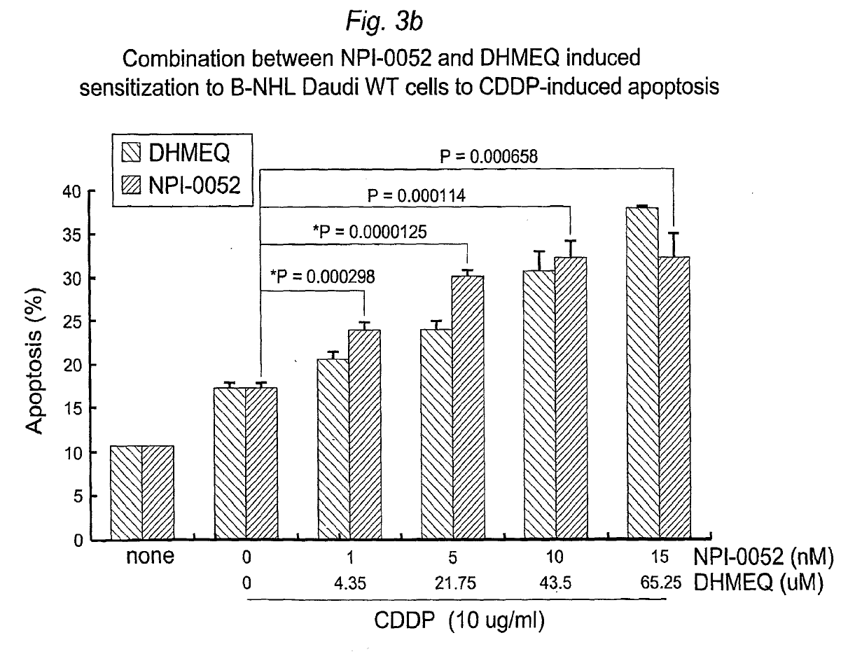

We examined the effect of Salinosporamide A and bortezomib in their ability to sensitize Daudi wild type cells to CDDP-induced apoptosis. The tumor cells were treated for one hour with various concentrations of Salinosporamide A or bortezomib (range 1-15 nM) and then treated with CDDP (10 μg/ml) for an additional 20 h. The cells were then examined for apoptosis as described above. The findings shown in FIG. 3A demonstrate that both agents yielded comparable results with respect to chemosensitization and with equivalent concentration dependent effects. There were significant apoptosis by the combination treatment at all concentrations of inhibitors used.

b) Study with Daudi RR1

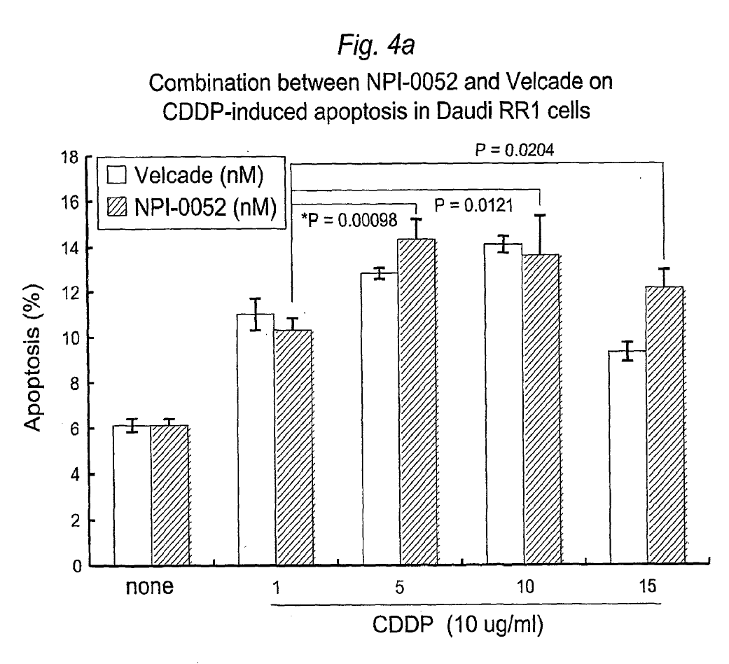

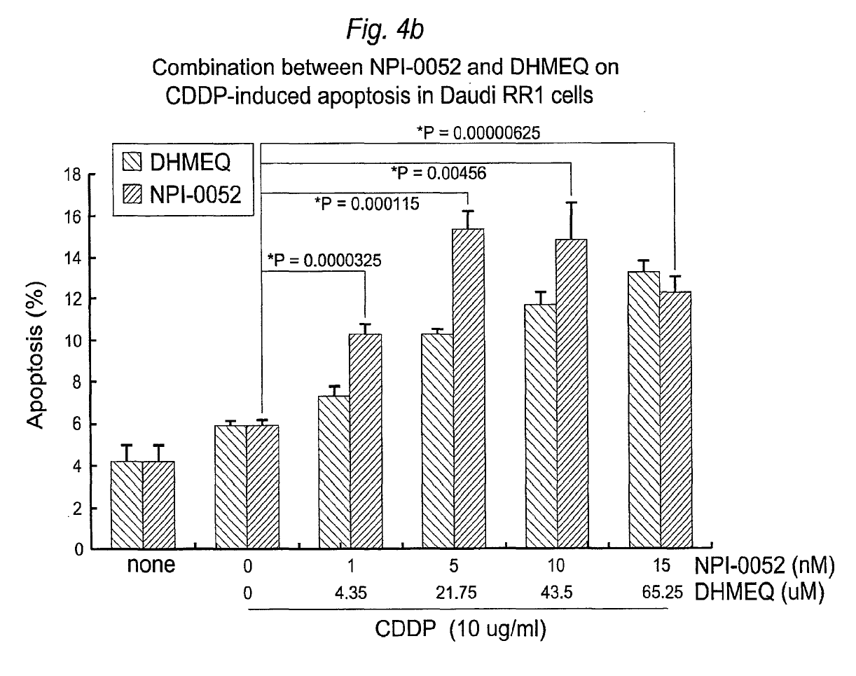

We performed similar experiments as above with rituximab resistant Daudi RR1 cells and the findings are summarized in FIG. 4A. Like Daudi wild type, significant potentiation of apoptosis was observed by both agents. In the combination treatment with CDDP, both inhibitors showed similar patterns of potentiation of cytotoxicity.

The findings with both Daudi and Daudi RR1 cells demonstrate that Salinosporamide A and bortezomib showed similar findings under the conditions used and the model system utilized. Further analysis by changing the time of treatment and with other cell lines will determine if there were differences with respect to the concentrations and cytotoxicity when using Salinosporamide A or bortezomib in sensitization experiments.

4) Comparison Between Salinosporamide A and the NF-κB Inhibitor, DHMEQ in their Ability To Sensitize Drug Resistant Tumor Cells to CDDP-Induced Apoptosis.

a) Study with Daudi Wild Type Cells

DHMEQ is a NF-κB inhibitor that has been shown to be selective and preventing NF-κB translocation from the cytoplasm to the nucleus (Horiguchi, et al., Expert Rev. Anticancer Ther., 2003, 3(6): 793-8.). We have reported that DHMEQ can sensitize drug-resistant tumor cells to drug-induced apoptosis. We examined the differential effects of Salinosporamide A and DHMEQ in their ability to sensitize Daudi to CDDP-induced apoptosis. Tumor cells were treated with different concentrations of DHMEQ (range 1-65 μM) and Salinosporamide A (range 1-50 nM) for 1 h and then treated with CDDP (10 μg/ml) for an additional 20 h and the cells were then examined for apoptosis as described above. The findings in FIG. 3B demonstrate that DHMEQ can sensitize Daudi cells to CDDP-induced apoptosis and sensitization was a function of the concentration used. Interestingly, the extent of sensitization by DHMEQ was similar to that obtained with Salinosporamide A, however, there was a significant difference in the amount of inhibitor used. A 4,000 fold higher concentration used with DHMEQ as compared to Salinosporamide A.

b) Study with Daudi RR1 Cells

Similar studies were performed as above in a) with Daudi RR1 cells. The findings in FIG. 4B demonstrate that both inhibitors sensitize cells to CDDP-induced apoptosis. Similar patterns were obtained by both inhibitors; however, there was a 4,000 fold higher concentration used with DHMEQ as compared to Salinosporamide A.

This finding demonstrates that Salinosporamide A is a superior inhibitor and sensitizing agent as compared to DHMEQ based on the concentration used. However, further studies are needed to demonstrate selectivity with other tumor cell lines.

### 5) Conclusions:

The above findings have demonstrated the following:

1) Salinosporamide A at very low concentrations (0.1-10 nM) sensitizes both rituximab sensitive and rituximab resistant B-NHL tumor cells to drug-induced apoptosis.

2) Comparing the effectiveness of Salinosporamide A and bortezomib in the model system used, revealed that both agents at similar concentrations sensitize B-NHL cells to drug-induced apoptosis.

3) Comparing Salinosporamide A and the specific NF-κB inhibitor DHMEQ revealed that both agents sensitized tumor cells to drug-induced apoptosis; however, sensitization by DHMEQ required a 4,000 fold increase in the concentration as compared to Salinosporamide A.

### Example II

### Salinosporamide A as a Chemotherapeutic Agent for Rituximab-Sensitized Cells

Our published work with B-NHL cells revealed that rituximab sensitized drug resistant tumor cells to drug induced apoptosis. Sensitization was the result of inhibition of survival pathways such as the Raf-Mek-Erk and NF-κB pathways. These pathways resulted to down regulation of the anti-apoptotic gene product, selectively Bclₓ₁ (Jazirehi and Bonavida, 2005). Since Salinosporamide A was shown to be cytotoxic in sensitive tumor cells, we considered that it might behave like a chemotherapeutic drug and thus we examined whether rituximab can sensitize tumor cells to Salinosporamide A induced apoptosis. We have reported that rituximab treatment of B-NHL cell lines sensitized the drug-resistant cells to drug-induced apoptosis. One of the mechanisms by which rituximab sensitizes the tumor cells to drug-induced apoptosis has been shown to be mediated via inhibition of the NF-κB pathway and downstream the selective inhibition of the anti-apoptotic product Bcl_{XL} expression. Inhibitors of this pathway mimicked rituximab in sensitizing the cells to drug-induced apoptosis (Jazirehi, et al, 2005, Cancer Research, 65(1):264-76). We hypothesized that proteasome inhibitors that inhibit NF-κB activity and downstream anti-apoptotic gene products may sensitize tumor cells to drug-induced apoptosis. The new proteasome inhibitor Salinosporamide A (Nereus Pharmaceuticals), which inhibits NF-κB activity, has been shown to sensitize B-NHL cells to drug (CDDP, adriamycin)-induced apoptosis. Salinosporamide A has also been shown to directly kill sensitive tumor cells by apoptosis. Also, Salinosporamide A induces apoptosis in multiple myeloma cells resistant to conventional and bortezomib therapies (Chauhan et al., Cancer Cell 2005 In Press).

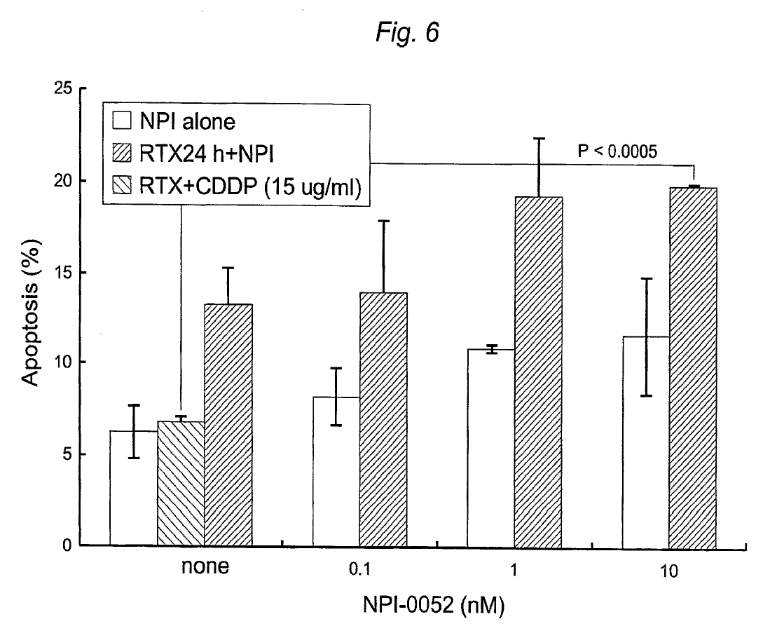

We hypothesized that Salinosporamide A may behave like a chemotherapeutic drug and rituximab may therefore sensitize the tumor cells to Salinosporamide A-induced apoptosis. Ramos cells were treated with rituximab (20 ug/ml) (12 h to 18 h) and the cells were treated with various concentrations of Salinosporamide A (1-10 nM) for an additional 20 h and the cells were examined for apoptosis using the PI method detecting DNA fragmentation by flow cytometry. The combination treatment resulted in significant apoptosis. The synergistic activity was detected with very low concentrations of Salinosporamide A>=1 nM. By comparison, several thousand fold higher concentrations of chemotherapeutic drugs (e.g. CDDP, adriamycin) were used for rituximab-mediated chemosensitization of Ramos cells. We also examined the Salinosporamide A resistant Daudi cells following treatment with rituximab for 1 h and Salinosporamide A for an additional 20 h and apoptosis was measured as before. The findings in FIG. 5 show that the combination of rituximab (20 ug/ml) and Salinosporamide A (10 nM) resulted in significant apoptosis and synergy was achieved. These findings demonstrate that the combination of rituximab and Salinosporamide A may be a therapeutic option for the treatment of drug-resistance and resistant tumor cells. FIG. 6 shows that in comparison to rituximab-mediated chemosensitization to CDDP, we have found that rituximab sensitizes to Salinosporamide A-induced apoptosis with a higher level than CDDP. The studies revealed that rituximab can sensitize drug-resistant tumor cells to Salinosporamide A induced apoptosis.

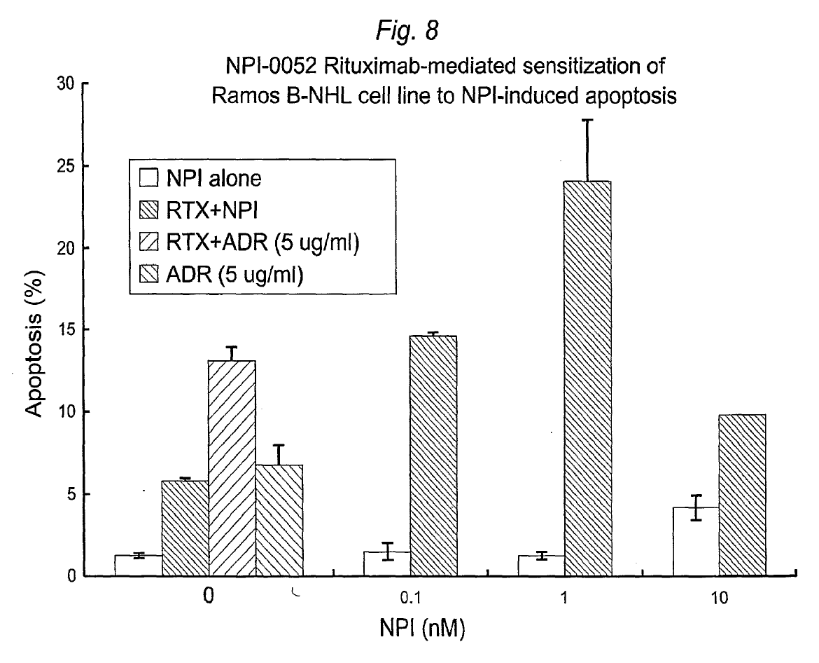

We further found that rituximab sensitizes cells to Salinosporamide A-induced apoptosis to a higher level than does adriamycin (ADR). As shown in FIG. 8, whereas sole treatment of cells with Salinosporamide A showed no detectable cytotoxic effects, pre-treatment of the tumor cells with rituximab resulted in significant sensitization of the tumor cells to Salinosporamide A-induced apoptosis and synergy was achieved. The sensitization of rituximab to Salinosporamide A-induced apoptosis was greater than that achieved with ADR. FIG. 8 also shows that low concentrations of 1 nM Salinosporamide A-induced significant apoptosis in rituximab pre-treated tumor cells. Higher concentrations of Salinosporamide A (10 nM) appear to be less effective due to cell loss.

These results demonstrate that rituximab sensitizes the tumor cells to the proteasome inhibitor Salinosporamide A-mediated apoptosis. In addition, the findings suggest that rituximab (or chemo sensitizing agents) used in combination with Salinosporamide A may result in synergistic activity and can reverse drug and/or rituximab resistance of B-NHL.

### Example Iii

### Salinosporamide a Induction of Raf-Kinase Inhibitor Protein (RKIP)

The acquisition of resistance to conventional therapies such as chemotherapy, radiation, and immunotherapy remains a major obstacle in the successful treatment of cancer. Among the mechanisms of resistance is the acquisition of resistance to apoptotic stimuli by tumor cells. Hence, tumor cells develop mechanisms to resist apoptosis and exhibit constitutive hyperactivation of survival and anti-apoptotic signaling pathways. Tumor suppressors exist in normal cells that negatively regulate cell survival and enhance response to apoptotic stimuli. The dysregulation of such controls that regulate cell survival and proliferation leads to neoplastic transformation. Thus, most tumor cells have dysregulated expression or function of functional tumor suppressors through deletion or mutation or low expression. Therefore, agents that can upregulate the expression of functional tumor suppressors would be useful to counteract the survival and anti-apoptotic pathways in tumor cells. Such agents would be expected to inhibit tumor cell proliferation or survival and/or sensitize cells to the cytotoxic effect of conventional cytotoxic therapies.

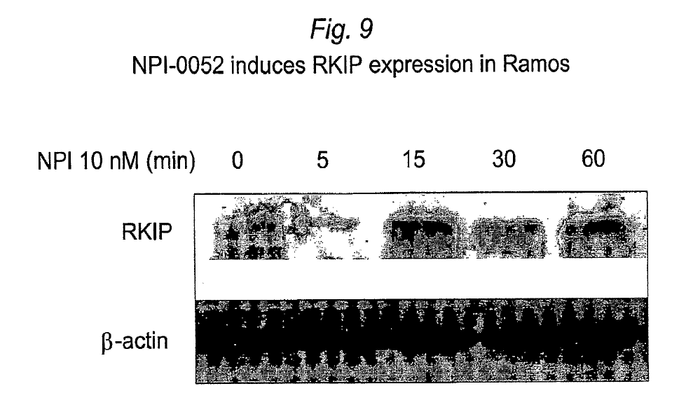

As shown in FIG. 9, treatment of tumor cells with Salinosporamide A results in the induction of the tumor suppressor, Raf-kinase inhibitor protein (RKIP). RKIP is a member of the phosphotidylethanolamine-binding protein (PEBP) family. Ramos cells were treated with 10 nM Salinosporamide A for various periods of time, and total cell lysates were examined for the expression of RKIP by western blot analysis. The expression of β-actin was used as a control. The data in FIG. 9 shows that there is an increase in the levels of RKIP protein after a 15 minute exposure of Ramos cells to 10 nM Salinosporamide A.

It has been shown that RKIP inhibits the Raf/MEK/ERK 1/2 and the NF-κB survival signaling pathways, and consequently, the expression of several anti-apoptotic gene products that are regulated by these pathways. Furthermore, expression of RKIP has been shown to reverse the resistance of drug-resistant cancer cells to drug-induced apoptosis. In addition, RKIP expression has been found to be depressed in primary tumors as compared to normal tissues and has been found to be lost following malignancy and metastasis in tumors. Thus, the ability of Salinosporamide A to induce the expression of RKIP in tumor cells provides a novel therapeutic target for avoiding or reversing therapy resistance of cancer cells and may be especially useful in treating metastases.

### Example IV

### Salinosporamide A Inhibits the Expression of YY1

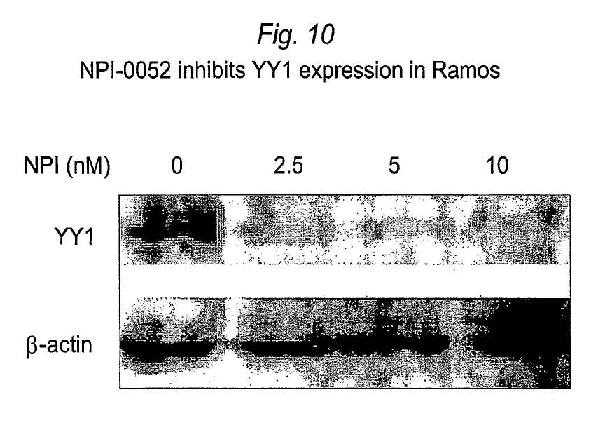

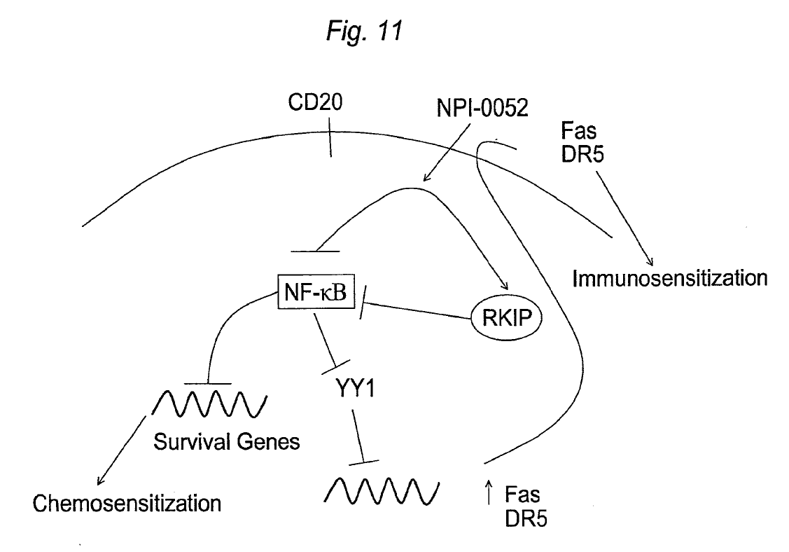

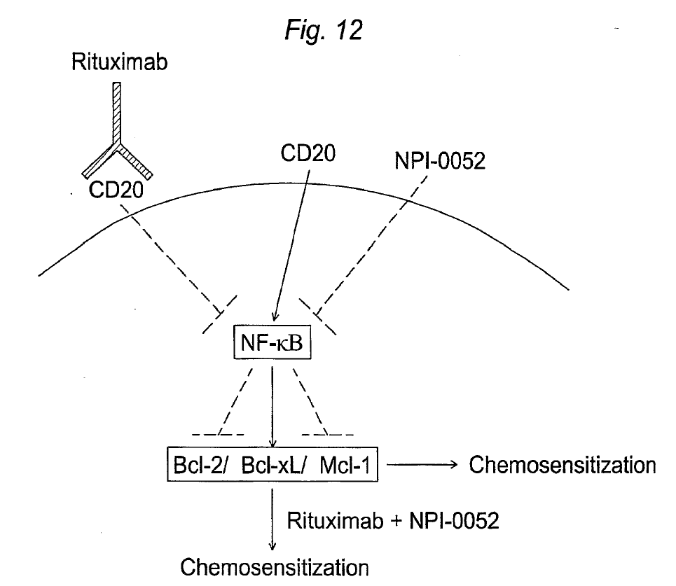

As shown in FIG. 10, treatment of tumor cells with Salinosporamide A results in the inhibition of expression of the transcriptional regulator protein YY1. YY1 is a transcription repressor that is overexpressed in cancer cells and has been shown to play a role in maintaining the resistance of tumor cells to various therapeutics. Ramos cells were treated with various concentrations of Salinosporamide A for 24 hours, and total cell lysates were examined for the expression of YY1 by western blot analysis and by RT-PCR. The expression of β-actin was used as a control. The data in FIG. 10 shows that there is a decrease in the levels of YY1 protein after a 24 hour exposure of Ramos cells to various concentrations of Salinosporamide A.

### Example V

### Salinosporamide A Analogs

Analogs of Salinosporamide A, as shown in Formula I and in US 20050049294 are tested for activity as sensitizing agents and as chemotherapeutic agents as described above in Examples I, II, and III.

### Example VI

### Salinosporamide A Activates Caspase 9 and Inhibits Bcl-xL Expression

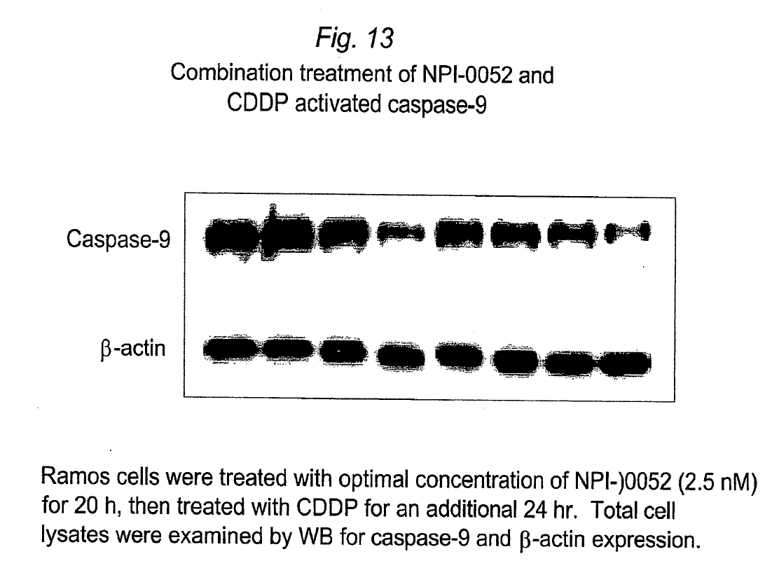

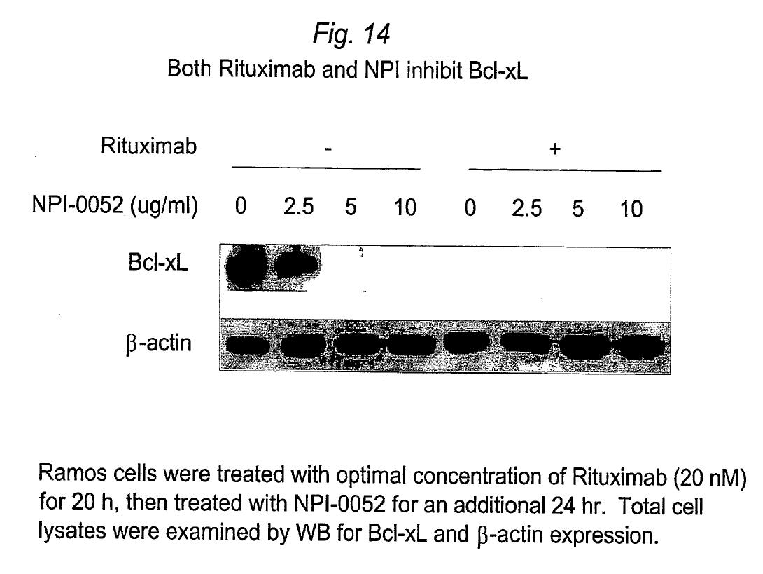

FIGS. 13 and 14 demonstrate that Salinosporamide A treatment of tumor cells results in the activation of caspase 9, and also inhibits expression of the anti-apoptotic gene BCLxl. Activation of caspase 9 indicates that Salinosporamide A activates the mitochondria and type II apoptosis and facilitates its direct or indirect activation of the effector caspases 3 and 7 for apoptosis. The inhibition of BCLxl by Salinosporamide A demonstrates that it inhibits a key anti-apoptotic factor that regulates resistance in many tumors. Also, it suggests that Salinosporamide A mediated inhibition of BCLxl may be responsible, in part, for its sensitizing effect on resistant tumor cells.

### Example VII

### Salinosporamide A Induces PTEN and Inhibits PTN

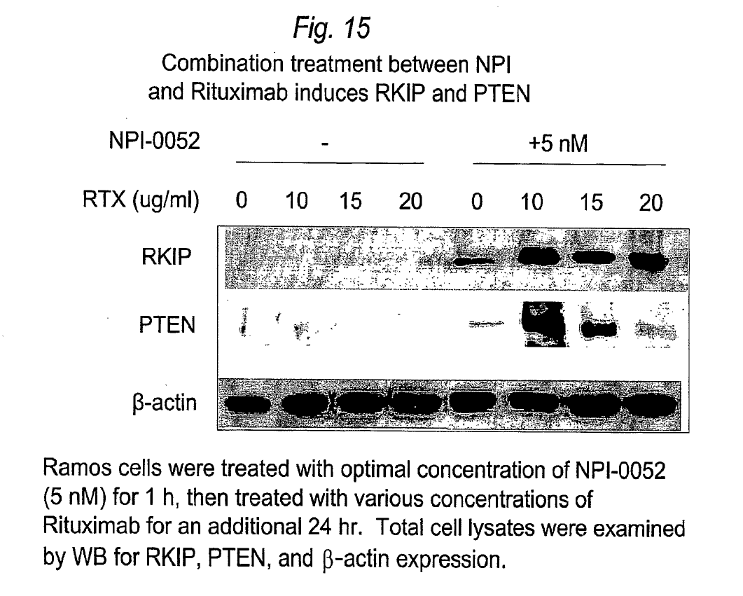

The findings in FIG. 15 demonstrate that Salinosporamide A induces expression of the phosphatase inhibitor PTEN, or the AKT cell survival pathway. This induction inhibits the anti-apoptotic AKT pathway and contributes to Salinosporamide A induced sensitization to apoptosis. Further, most resistant tumor cells express low levels of PTEN, and PTEN is a target for therapeutic intervention, as well as a biomarker

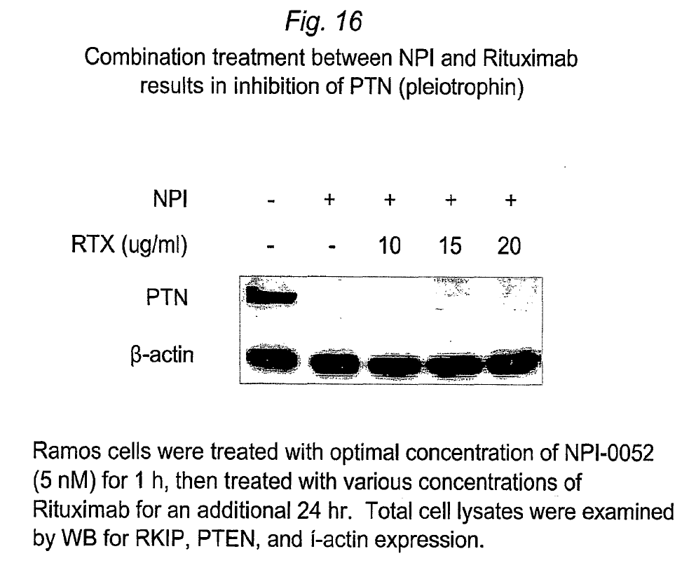

The findings in FIG. 16 demonstrate that Salinosporamide A inhibits the expression of the growth factor pleiotrophin (PTN). PTN has been reported to be elevated in tumor cells and in the circulation of cancer patients and is of prognostic significance. In addition, inhibition of PTN contributes to the sensitizing effect of Salinosporamide A. PTN is also a target for therapeutic intervention.

### Example VII

### Salinosporamide A Upregulates Death Receptor DR5 and Sensitizes Tumor Cells to TRAIL-Induced Apoptosis

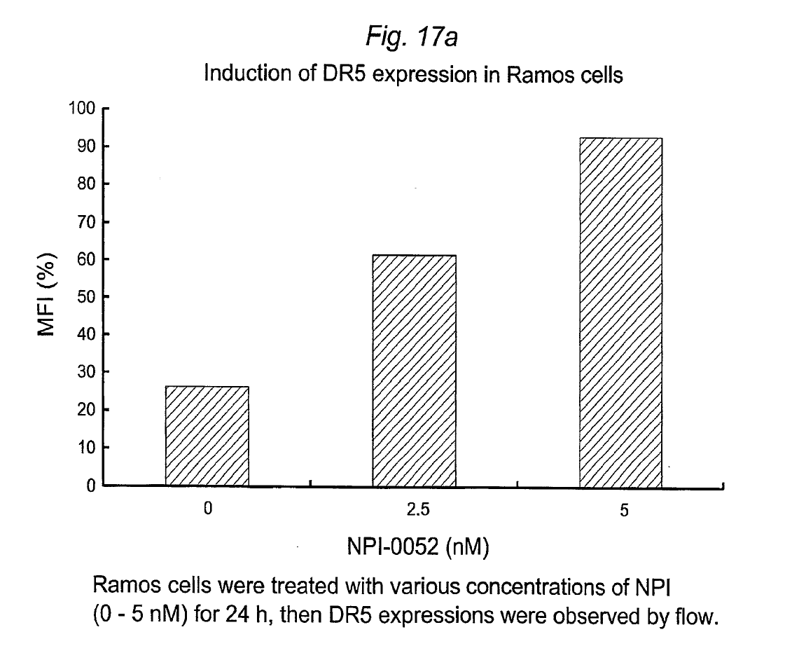

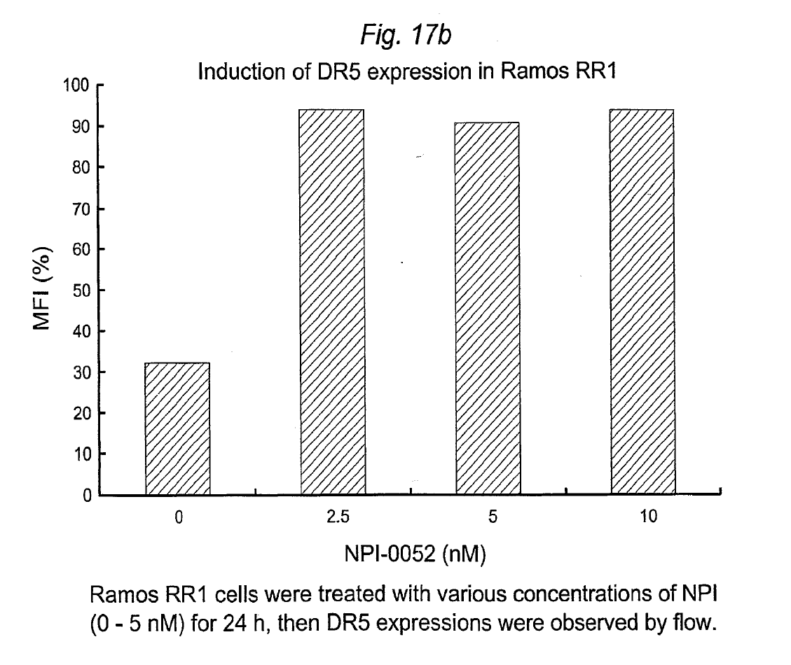

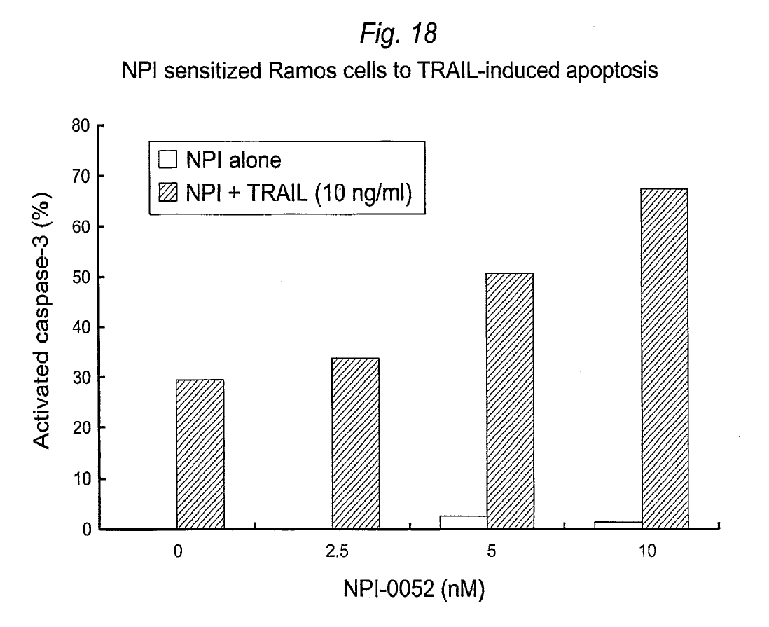

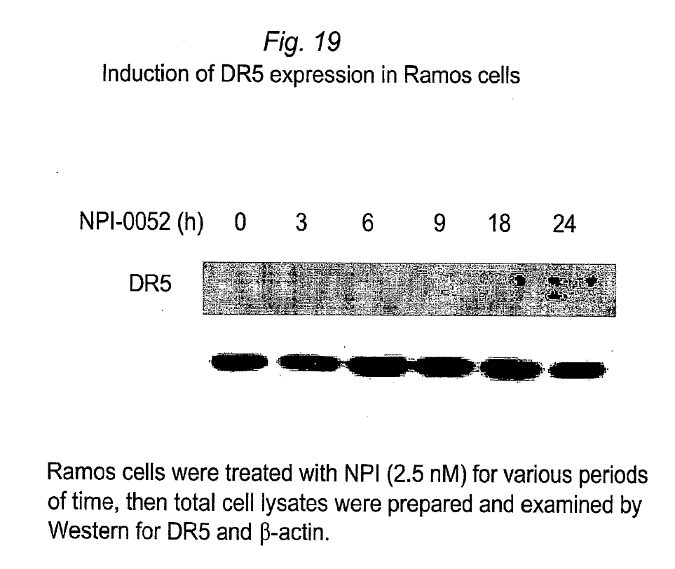

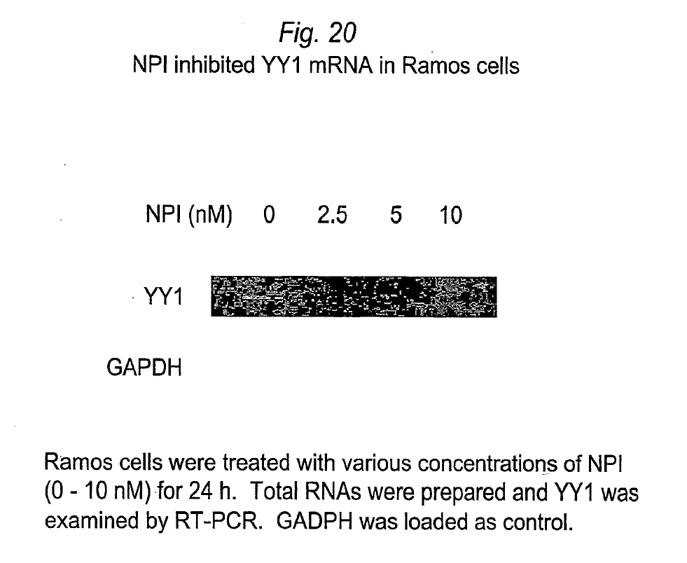

FIGS. 17 A and B, 18 and 19 demonstrate that Salinosporamide A upregulates the expression of the TRAIL death receptor DR5 and sensitizes TRAIL resistant tumor cells to TRAIL mediated apoptosis. These findings demonstrate also that Salinosporamide A is a therapeutic agent that can be used in combination with TRAIL or agonist anti-DR5/DR5 mAbs in the treatment of drug/TRAIL resistant cells.

### REFERENCES

Feling, et al., Angew. Chem. Int. Ed., 2003, 42(3): 355-357
Horiguchi, et al., Expert Rev. Anticancer Ther., 2003, 3(6): 793-8.
Jazirehi and Bonavida, Oncogene. 2005, 24(13):2121-43.
Macherla, et al., Journal of Medicinal Chemistry, 2005, 48:3684
Suzuki, et al., AACR Annual Meeting, Abstract number 5429, Apr. 1-5, 2006.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of treating, preventing or inhibiting a cancer, the method comprising the step of administering to a subject a therapeutically effective amount of a cancer therapy reagent and a sensitizingly effective amount of a compound of Formula I: 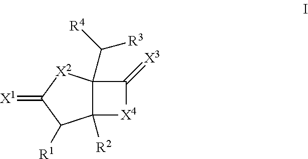
wherein
each of R¹, R² and R³ are independently selected from the group consisting of H, alkyl, substituted alkyl, alkenyl, substituted alkenyl, alkynyl, substituted alkynyl, aryl, substituted aryl, heteroaryl, substituted heteroaryl, heterocyclic, substituted heterocyclic, cycloalkyl, substituted cycloalkyl, alkoxy, substituted alkoxy, thioalkyl, substituted thioalkyl, hydroxy, halogen, amino, amido, carboxyl, —C(O)H, acyl, oxyacyl, carbamate, sulfonyl, sulfonamide, and sulfuryl;
R⁴ is a 5-8 membered cycloalkyl optionally substituted with 1-8 R⁵ groups or a 5-8 membered cycloalkenyl optionally substituted with 1-8 R⁵ groups;
each R⁵ is independently selected from the group consisting of alkyl, substituted alkyl, alkenyl, substituted alkenyl, alkynyl, substituted alkynyl, aryl, substituted aryl, cycloalkyl, and substituted cycloalkyl;
each of X¹, X², X³ and X⁴ is independently selected from the group consisting of O, NR⁶ and S; and
R⁶ is H or C₁-C₆ alkyl.

2. The method of claim 1, wherein each of R¹ and R² are independently selected from the group consisting of H, alkyl, substituted alkyl, alkenyl, substituted alkenyl, alkynyl, substituted alkynyl, cycloalkyl, and substituted cycloalkyl;
R³ is selected from the group consisting of alkoxy, substituted alkoxy, thioalkyl, substituted thioalkyl, hydroxy, halogen, amino, amido, oxyacyl, carbamate, sulfonyl, sulfonamide, and sulfuryl;
R⁴ is a 5-8 membered cycloalkenyl optionally substituted with 1-8 R⁵ groups; each of X¹, X³ and X⁴ is O; and
X² is NH.

3. The method of claim 1, wherein each of R¹ and R² are independently selected from the group consisting of alkyl, substituted alkyl, alkenyl, substituted alkenyl, alkynyl, and substituted alkynyl;
R³ is selected from the group consisting of alkoxy, substituted alkoxy, thioalkyl, substituted thioalkyl, hydroxy, and amino;
R⁴ is cyclohexenyl optionally substituted with 1-8 R⁵ groups;
each of X¹, X³ and X⁴ is O; and X² is NH.

4. The method of claim 1, wherein
R¹ is an alkyl or substituted alkyl;
R² is alkyl;
R³ is hydroxy;
R⁴ is cyclohexenyl; and
each of X¹, X³ and X⁴ is O; and
X² is NH.

5. The method of claim 4, wherein the substituted alkyl of R¹ is a halogenated alkyl.

6. The method of claim 5, wherein the halogenated alkyl is selected from the group consisting of a fluorinated alkyl, chlorinated alkyl, and brominated alkyl.

7. (canceled)

8. (canceled)

9. The method of claim 7, wherein the compound has the following structure:

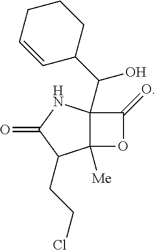

10. The method of claim 7, wherein the compound has the following structure:

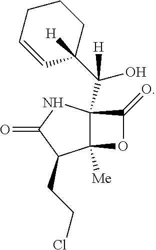

11. The method of claim 1, wherein the sensitizingly effective amount of the compound of Formula I is sufficient to inhibit expression of an anti-apoptotic gene product, thereby inducing apoptosis.

12. (canceled)

13. (canceled)

14. (canceled)

15. (canceled)

16. (canceled)

17. (canceled)

18. (canceled)

19. (canceled)

20. The method of claim 1, wherein the cancer therapy reagent is a chemotherapeutic reagent, an immunotherapeutic reagent, a radiotherapeutic reagent, or a hormonal therapeutic reagent.

21. The method of claim 1, wherein the cancer is selected from the group consisting of: non-Hodgkin's lymphoma, B-acute lymphoblastic lymphoma, prostate cancer, ovarian cancer, renal cancer, lung cancer, breast cancer, colon cancer, leukemia, multiple myeloma and hepatocarcinoma.

22. The method of claim 21, wherein the cancer is lymphoma.

23. The method of claim 21, wherein the cancer is non-Hodgkin's lymphoma.

24. The method of claim 1, wherein the cancer therapy reagent induces apoptosis.

25. The method of claim 24, wherein the cancer therapy reagent is a chemotherapeutic reagent, an immunotherapeutic reagent, a radiotherapeutic reagent, or a hormonal therapeutic reagent.

26. The method of claim 24, wherein the cancer therapy reagent is rituximab immunotherapy.

27. The method of claim 26, wherein the cancer is B-NHL.

28. (canceled)

29. The method of claim 1, wherein the cancer is therapy-resistant.

30. The method of claim 29, wherein the therapy is selected from the group consisting of immunotherapy, chemotherapy, radiotherapy, and hormonal therapy.

31. The method of claim 1, wherein the cancer is therapy-sensitive.

32. The method of claim 1, wherein the therapeutically effective amount of a cancer therapy reagent and the sensitizingly effective amount of a compound of Formula I are administered concurrently.

33. The method of claim 32, wherein the cancer therapy reagent comprises bortezomib administration.

34. The method of claim 32, wherein the cancer therapy reagent is a chemotherapeutic reagent, an immunotherapeutic reagent, a radiotherapeutic reagent, or a hormonal therapeutic reagent.

35. The method of claim 1, wherein the therapeutically effective amount of a cancer therapy reagent and the sensitizingly effective amount of a compound of Formula I are administered sequentially.

36. The method of claim 1, wherein the subject is a human.

37. (canceled)

38. (canceled)

39. (canceled)

40. (canceled)

41. (canceled)

42. (canceled)

43. A composition comprising a therapeutically effective amount of rituximab and a sensitizingly effective amount of a compound of Formula I in a physiologically acceptable excipient.

44. A kit comprising a therapeutically effective amount of rituximab and a sensitizingly effective amount of a compound of Formula I.

45. A method of treating, preventing or inhibiting a cancer with proteasome inhibitor therapy, the method comprising the step of administering to a subject a sensitizingly effective amount of an antibody or chemosensitizing reagent and a therapeutically effective amount of a compound of Formula I: 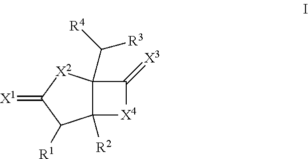
wherein
each of R¹, R² and R³ are independently selected from the group consisting of H, alkyl, substituted alkyl, alkenyl, substituted alkenyl, alkynyl, substituted alkynyl, aryl, substituted aryl, heteroaryl, substituted heteroaryl, heterocyclic, substituted heterocyclic, cycloalkyl, substituted cycloalkyl, alkoxy, substituted alkoxy, thioalkyl, substituted thioalkyl, hydroxy, halogen, amino, amido, carboxyl, —C(O)H, acyl, oxyacyl, carbamate, sulfonyl, sulfonamide, and sulfuryl;
R⁴ is a 5-8 membered cycloalkyl optionally substituted with 1-8 R⁵ groups or a 5-8 membered cycloalkenyl optionally substituted with 1-8 R⁵ groups;
each R⁵ is independently selected from the group consisting of alkyl, substituted alkyl, alkenyl, substituted alkenyl, alkynyl, substituted alkynyl, aryl, substituted aryl, cycloalkyl, and substituted cycloalkyl;
each of X¹, X², X³ and X⁴ is independently selected from the group consisting of O, NR⁶ and S; and
R⁶ is H or C₁-C₆ alkyl.

46. (canceled)

47. The method of claim 45, wherein the antibody is rituximab.

48. The method of claim 45, wherein the cancer is lymphoma.

49. A composition comprising a sensitizingly effective amount of rituximab and a therapeutically effective amount of a compound of Formula I in a physiologically acceptable excipient.

50. A kit comprising a sensitizingly effective amount of rituximab and a therapeutically effective amount of a compound of Formula I.

51. A method of treating, preventing or inhibiting a cancer, the method comprising the step of administering to a subject a therapeutically effective amount of a compound of Formula I: 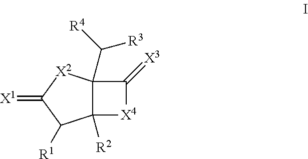
wherein
each of R² and R³ are independently selected from the group consisting of H, alkyl, substituted alkyl, alkenyl, substituted alkenyl, alkynyl, substituted alkynyl, aryl, substituted aryl, heteroaryl, substituted heteroaryl, heterocyclic, substituted heterocyclic, cycloalkyl, substituted cycloalkyl, alkoxy, substituted alkoxy, thioalkyl, substituted thioalkyl, hydroxy, halogen, amino, amido, carboxyl, —C(O)H, acyl, oxyacyl, carbamate, sulfonyl, sulfonamide, and sulfuryl;
R⁴ is a 5-8 membered cycloalkyl optionally substituted with 1-8 R⁵ groups or a 5-8 membered cycloalkenyl optionally substituted with 1-8 R⁵ groups;
each R⁵ is independently selected from the group consisting of alkyl, substituted alkyl, alkenyl, substituted alkenyl, alkynyl, substituted alkynyl, aryl, substituted aryl, cycloalkyl, and substituted cycloalkyl;
each of X¹, X², X³ and X⁴ is independently selected from the group consisting of O, NR⁶ and S; and
R⁶ is H or C₁-C₆ alkyl, and
wherein said therapeutically effective amount is sufficient to induce the expression of RKIP or PTEN, thereby inducing apoptosis.

52. (canceled)

53. (canceled)

54. (canceled)

55. (canceled)

56. (canceled)

57. (canceled)

58. A method of treating a therapy resistant cancer, the method comprising the step of administering to a subject a therapeutically effective amount of a compound of Formula I: 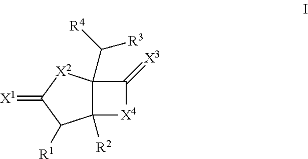
wherein
each of R¹, R² and R³ are independently selected from the group consisting of H, alkyl, substituted alkyl, alkenyl, substituted alkenyl, alkynyl, substituted alkynyl, aryl, substituted aryl, heteroaryl, substituted heteroaryl, heterocyclic, substituted heterocyclic, cycloalkyl, substituted cycloalkyl, alkoxy, substituted alkoxy, thioalkyl, substituted thioalkyl, hydroxy, halogen, amino, amido, carboxyl, —C(O)H, acyl, oxyacyl, carbamate, sulfonyl, sulfonamide, and sulfuryl;
R⁴ is a 5-8 membered cycloalkyl optionally substituted with 1-8 R⁵ groups or a 5-8 membered cycloalkenyl optionally substituted with 1-8 R⁵ groups;
each R⁵ is independently selected from the group consisting of alkyl, substituted alkyl, alkenyl, substituted alkenyl, alkynyl, substituted alkynyl, aryl, substituted aryl, cycloalkyl, and substituted cycloalkyl;
each of X¹, X², X³ and X⁴ is independently selected from the group consisting of O, NR⁶ and S; and
R⁶ is H or C₁-C₆ alkyl, and wherein said therapeutically effective amount is sufficient to induce the expression of RKIP or PTEN, thereby inducing apoptosis.
